(19) United States
(12) Patent Application Publication
(10) Pub. No.: US 2025/0353014 A1
(43) Pub. Date: Nov. 20, 2025

(54) DEVICES AND METHODS FOR PLATELET ASSAY

(71) Applicant: Essenlix Corporation, Monmouth Junction, NJ (US)

(72) Inventors: Stephen Y. Chou, Princeton, NJ (US); Wei Ding, Princeton, NJ (US); Ji Qi, Hillsborough, NJ (US); Jun Tian, Belle Mead, NJ (US); Yuecheng Zhang, Yardley, PA (US)

(73) Assignee: Essenlix Corporation, Monmouth Junction, NJ (US)

(21) Appl. No.: 19/283,097

(22) Filed: Jul. 28, 2025

Related U.S. Application Data
(63) Continuation of application No. 16/640,312, filed on Feb. 19, 2020, now abandoned.
(60) Provisional application No. 62/539,672, filed on Aug. 1, 2017.

(51) Int. Cl. B01L 3/00; G01N 1/28; G01N 15/10; G01N 15/14; G01N 33/50

(52) U.S. Cl. CPC B01L 3/50853; B01L 3/5088; G01N 1/2813; G01N 1/286; G01N 15/1468; G01N 33/5002; B01L 2300/041; B01L 2300/0822; B01L 2400/086; G01N 2015/1006; G01N 2015/1486

(57) ABSTRACT

The present invention provides devices, systems, and methods for performing biological and chemical assays.

### CROSS-REFERENCE

This application is a continuation application of U.S. non-provisional application Ser. No. 16/640,312, filed on Feb. 19, 2020, which is a National Stage entry (§ 371) application of International Application No. PCT/US2018/044865, filed on Aug. 1, 2018, which claims the benefit of U.S. Provisional Patent Application No. 62/539,672, filed Aug. 1, 2017, the contents of which are relied upon and incorporated herein by reference in their entirety.

### FIELD

Among other things, the present invention is related to devices and methods of performing biological and chemical assays, in particular, of platelets.

### BACKGROUND

In biological and chemical assays, it is often difficult and inaccurate in viewing platelets in undiluted or slightly diluted whole blood (with the most cells un-lysed). This is because, due to the relatively small size of platelets, certain cells in a whole blood can block or disrupt a clear viewing and/counting of the platelets. One example of these cells are red blood cells, which are much larger than platelets and can attenuate an optical signal.

The present invention provides devices and methods for improved viewing and/or counting of the platelets in undiluted or slightly diluted whole blood, or other types of blood sample.

One aspect of the present invention uses (a) two plates to compress a whole blood sample into a thin layer that has a thickness and lyses the red cells, and (b) after (a), imaging process to view and/or counting the platelets. Spacers are used to control the final sample thickness and hence to assist a determination of the platelet concentration.

Another aspect of the present invention provides uniformity of gap size between the two plates, hence leading to uniform lysing of specific cell types (e.g. red blood cells) over a significant area.

Another aspect of the present invention is to selectively lyse one type of cells (e.g. red blood cells and/or white blood cells) in a blood sample, while platelets in the sample are left un-lysed.

Another aspect of the present invention is to use reagent coated on the surface of one or both of the plates to facilitate the lysing of red blood cells and/or white blood cells in the sample, and/or the unlysing of the platelets.

Another aspect of the present invention is to use imaging technique to view/count the platelets in the sample in bright-filed mode and/or fluorescent mode.

Another aspect of the present invention is to use mobile communication device to facilitate the imaging and counting, and in some cases, remote health monitoring of the user of the devices.

### BRIEF DESCRIPTION OF THE DRAWINGS

The skilled artisan will understand that the drawings, described below, are for illustration purposes only. The drawings are not intended to limit the scope of the present teachings in any way. Some of the drawings are not in scale. In the figures that present experimental data points, the lines that connect the data points are for guiding a viewing of the data only and have no other means.

FIG. 1 shows an embodiment of a generic QMAX (Q: quantification; M: magnifying; A: adding reagents; X: acceleration; also known as compressed regulated open flow (CROF)) device.

FIG. 2 shows an exemplary embodiment of the device and method provided by the present invention for platelet analysis, illustrating a general procedure of processing, imaging, and analyzing a blood sample.

FIG. 3 shows exemplary embodiments of the device and method for platelet analysis as provided by the present invention, which mechanically lyse red blood cells and optionally white blood cells in a selective manner for improved viewing and imaging of platelet in blood sample.

FIG. 4 shows an exemplary embodiment of the device and method for platelet analysis as provided by the present invention, which selectively lyse RBCs and WBCs using chemicals stored on the plate(s).

### DETAILED DESCRIPTION OF EXEMPLARY EMBODIMENTS

The following detailed description illustrates some embodiments of the invention by way of example and not by way of limitation. If any, the section headings and any subtitles used herein are for organizational purposes only and are not to be construed as limiting the subject matter described in any way. The contents under a section heading and/or subtitle are not limited to the section heading and/or subtitle, but apply to the entire description of the present invention.

The citation of any publication is for its disclosure prior to the filing date and should not be construed as an admission that the present claims are not entitled to antedate such publication by virtue of prior invention. Further, the dates of publication provided can be different from the actual publication dates which need to be independently confirmed.

Among other things, the present invention provides devices, systems, and methods of performing biological and chemical assays using a QMAX card.

The exemplary embodiments herein disclosed can be combined with the bio/chemical devices and assays including, but not limited to, the devices and assays as disclosed, described, and/or referred to in the following applications:
PCT Application No. PCT/US2016/045437, which was filed on Aug. 10, 2016,
PCT Application No. PCT/US2016/051775, which was filed on Sep. 14, 2016,
PCT Application No. PCT/US2016/051794, which was filed on Sep. 14, 2016,
U.S. Provisional Application No. 62/369,181, which was filed on Jul. 31, 2016,
U.S. Provisional Application No. 62/412,006, which was filed on Oct. 24, 2016,
U.S. Provisional Application No. 62/437,339, which was filed on Dec. 21, 2016,
U.S. Provisional Application No. 62/431,639, which was filed on Dec. 9, 2016,
U.S. Provisional Application No. 62/456,065, which was filed on Feb. 7, 2017,
U.S. Provisional Application No. 62/456,488, which was filed on Feb. 8, 2017,
U.S. Provisional Application No. 62/456,287, which was filed on Feb. 8, 2017,
U.S. Provisional Application No. 62/456,528, which was filed on Feb. 8, 2017,
U.S. Provisional Application No. 62/456,537, which was filed on Feb. 8, 2017,
U.S. Provisional Application No. 62/456,612, which was filed on Feb. 8, 2017,
U.S. Provisional Application No. 62/456,631, which was filed on Feb. 8, 2017,
U.S. Provisional Application No. 62/456,596, which was filed on Feb. 8, 2017,
U.S. Provisional Application No. 62/456,590, which was filed on Feb. 8, 2017,
U.S. Provisional Application No. 62/456,638, which was filed on Feb. 8, 2017,
U.S. Provisional Application No. 62/456,598, which was filed on Feb. 8, 2017,
U.S. Provisional Application No. 62/456,552, which was filed on Feb. 8, 2017,
U.S. Provisional Application No. 62/456,603, which was filed on Feb. 8, 2017,
U.S. Provisional Application No. 62/456,585, which was filed on Feb. 8, 2017,
U.S. Provisional Application No. 62/456,628, which was filed on Feb. 8, 2017,
U.S. Provisional Application No. 62/456,504, which was filed on Feb. 8, 2017,
U.S. Provisional Application No. 62/456,988, which was filed on Feb. 9, 2017,
U.S. Provisional Application No. 62/457,084, which was filed on Feb. 9, 2017,
U.S. Provisional Application No. 62/457,031, which was filed on Feb. 9, 2017,
U.S. Provisional Application No. 62/456,904, which was filed on Feb. 9, 2017,
U.S. Provisional Application No. 62/457,075, which was filed on Feb. 9, 2017,
U.S. Provisional Application No. 62/457,009, which was filed on Feb. 9, 2017,
U.S. Provisional Application No. 62/457,133, which was filed on Feb. 9, 2017,
U.S. Provisional Application No. 62/457,103, which was filed on Feb. 9, 2017,
U.S. Provisional Application No. 62/459,267, which was filed on Feb. 15, 2017,
U.S. Provisional Application No. 62/459,303, which was filed on Feb. 15, 2017,
U.S. Provisional Application No. 62/459,337, which was filed on Feb. 15, 2017,
U.S. Provisional Application No. 62/459,232, which was filed on Feb. 15, 2017,
U.S. Provisional Application No. 62/459,160, which was filed on Feb. 15, 2017,
U.S. Provisional Application No. 62/459,972, which was filed on Feb. 16, 2017,
U.S. Provisional Application No. 62/394,753, which was filed on Sep. 15, 2016,
U.S. Provisional Application No. 62/459,496, which was filed on Feb. 15, 2017,
U.S. Provisional Application No. 62/459,554, which was filed on Feb. 15, 2017,
U.S. Provisional Application No. 62/460,047, which was filed on Feb. 16, 2017,
U.S. Provisional Application No. 62/459,598, which was filed on Feb. 15, 2017,
U.S. Provisional Application No. 62/460,083, which was filed on Feb. 16, 2017,
U.S. Provisional Application No. 62/460,076, which was filed on Feb. 16, 2017,
U.S. Provisional Application No. 62/460,062, which was filed on Feb. 16, 2017,
U.S. Provisional Application No. 62/459,920, which was filed on Feb. 16, 2016,
U.S. Provisional Application No. 62/459,577, which was filed on Feb. 15, 2017,
U.S. Provisional Application No. 62/459,602, which was filed on Feb. 15, 2017,
U.S. Provisional Application No. 62/460,069, which was filed on Feb. 16, 2017,
U.S. Provisional Application No. 62/460,088, which was filed on Feb. 16, 2017,
U.S. Provisional Application No. 62/460,091, which was filed on Feb. 16, 2017,
U.S. Provisional Application No. 62/460,757, which was filed on Feb. 18, 2017,
U.S. Provisional Application No. 62/463,578, which was filed on Feb. 24, 2017,
which are all hereby incorporated in reference by their entireties.

The embodiments in these applications herein incorporated can be regarded in combination with one another or as a single invention, rather than as discrete and independent filings. Moreover, the exemplary embodiments disclosed herein are applicable to embodiments including but not limited to: bio/chemical assays, QMAX cards and systems, QMAX with hinges, notches, recessed edges and sliders, assays and devices with uniform sample thickness, smartphone detection systems, cloud computing designs, various detection methods, labels, capture agents and detection agents, analytes, diseases, applications, and samples; the various embodiments are disclosed, described, and/or referred to in the aforementioned applications, all of which are hereby incorporated in reference by their entireties.

The current invention relates to identifying, tracking, and/or monitoring of any device that can be imaged for certain analysis (e.g. bio/chemical assays). The QMAX card is disclosed

### QMAX Device

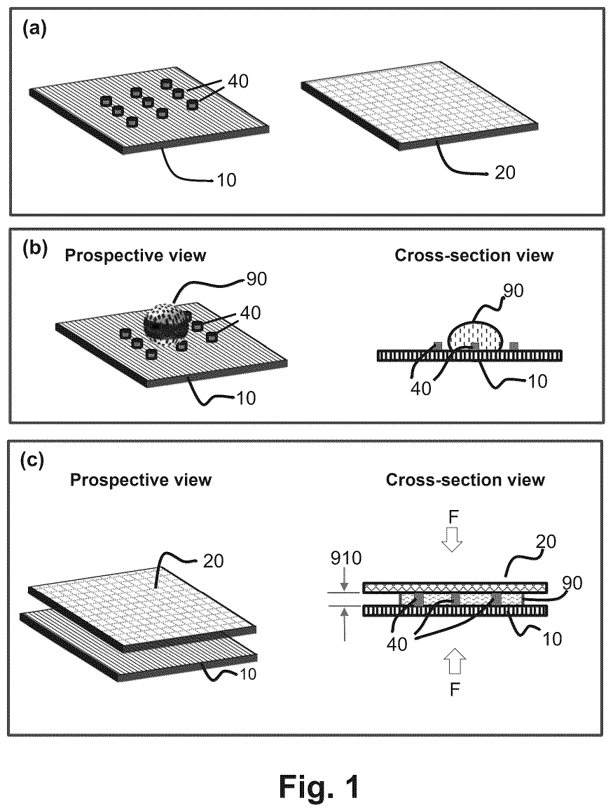

FIG. 1 shows an embodiment of a generic QMAX (Q: quantification; M: magnifying; A: adding reagents; X: acceleration; also known as compressed regulated open flow (CROF)) device. The generic QMAX device comprises a first plate 10 and a second plate 2. In particular, panel (A) shows the perspective view of a first plate 10 and a second plate 20 wherein the first plate has spacers. It should be noted, however, that the spacers can also be fixed on the second plate 20 (not shown) or on both first plate 10 and second plate 20 (not shown). Panel (B) shows the perspective view and a sectional view of depositing a sample 90 on the first plate 10 at an open configuration. It should be noted, however, that the sample 90 also can also be deposited on the second plate 20 (not shown), or on both the first plate 10 and the second plate 20 (not shown). Panel (C) illustrates (i) using the first plate 10 and second plate 20 to spread the sample 90 (the sample flow between the inner surfaces of the plates) and reduce the sample thickness, and (ii) using the spacers and the plate to regulate the sample thickness at the closed configuration of the QMAX device. The inner surfaces of each plate have one or a plurality of binding sites and or storage sites (not shown).

In some embodiments, the spacers 40 have a predetermined uniform height and a predetermined uniform inter-spacer distance. In the closed configuration, as shown in panel (C) of FIG. 1, the spacing between the plates and the thus the thickness of the sample 90 is regulated by the spacers 40. In some embodiments, the uniform thickness of the sample 90 is substantially similar to the uniform height of the spacers 40. It should be noted that although FIG. 1 shows the spacers 40 to be fixed on one of the plates, in some embodiments the spacers are not fixed. For example, in certain embodiments the spacers are mixed with the sample so that when the sample is compressed into a thin layer, the spacers, which is rigid beads or particles that have a uniform size, regulate the thickness of the sample layer.

### General Procedure

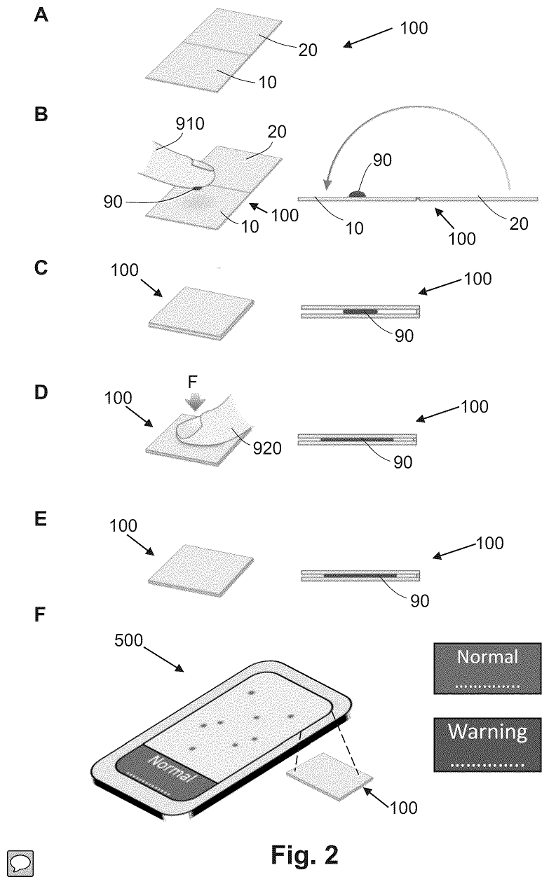

FIG. 2 shows an exemplary embodiment of the device and method provided by the present invention for platelet analysis. Panels (A) to (F) sequentially illustrate a general procedure using the exemplary QMAX device and system to identify and analyze platelets in a whole blood sample.

Panel (A) of FIG. 2 shows the QMAX device 100 for platelet assay, which comprises a first plate 10 and a second plate 20 that are connected to one another and capable of being open (as shown in panels (A) and (B)) and closed (panels (C)-(F)) like a book. Panel (B) shows that when the QMAX device 100 is open, a whole blood sample 90 is deposited onto the first plate 10. Here, shown as an example in the schematic on the left, the whole blood sample 90 is directly deposited from a pricked finger 910 to the first plate 10. It should be noted that, however, the sample can be deposited on either the first plate 10, the second plate 20, or both. The schematic on the right is a cross-sectional view of the QMAX device 100 bearing the blood sample 90. The curve arrow indicates the direction of folding the plates in order to bring them into a closed configuration.

Panels (C) to (E) of FIG. 2 illustrate the process of bringing the QMAX 100 from the open configuration to the closed configuration. Initially, the two plates 10 and 20 are brought to face each other with the blood sample 90 in between (C). Then, a compressing force F is applied to reduce the spacing between the two plates, spreading the sample 90 between the two plates (D). As an example, the compressing force F is applied through a finger 920 until the two plates enter the closed configuration as shown in panel (E).

It is one aspect of the present invention that the QMAX device is used to lyse the RBCs in the sample, facilitating the viewing and/or imaging of the platelets in the sample. Therefore, at the closed configuration, a substantial fraction of the RBCs, and in some embodiments, optionally, WBCs as well, are lysed in a relevant volume of the sample, while a substantial fraction of the platelets are not lysed.

As used herein, the term “substantial fraction” refers to a percentage equal to or more than 50%, 51%, 55%, 60%, 65%, 70%, 75%, 80%, 85%, 90%, 95%, 99% or 100%, or in a range between any of the two percentage values.

Lastly, as shown in panel (F) of FIG. 2, while the two plates are at the closed configuration, images of the platelets (symbolized by the green circles) between the two plates are acquired, for instance, through a mobile phone 500. Analysis of platelets is performed with the same phone 500 and readout of the analysis is given, as indicated by the green “Normal” sign or the red “Warning” sign.

### Selective Lysing

In some embodiments, the QMAX device selectively lyses the RBCs and optionally the WBCs through mechanical pressure, while leaving the platelets unlysed. In some embodiments, the QMAX device lyses the RBCs and optionally the WBCs through chemical reagent contained in the QMAX device, while leaving the platelets unlysed. In some embodiments, the QMAX device lyses the RBCs and optionally the WBCs through a combination of mechanical pressure provided thereby and chemical reagents contained therein and/or pre-loaded in the sample.

### 1. Mechanical Lysing

In some embodiments, the two plates are used to apply mechanical force against the cells contained in the sample that is deposited between the two plates, while the two plates are compressed to enter the closed configuration. If the spacing between the two plates at the closed configuration is smaller than the natural dimension of the cells in the sample between the plates, the two plates are likely to press against and deform the cells. The deformation creates an increased internal pressure against the cell enclosure, and when such an increased internal pressure exceeds the tolerable threshold of the cell enclosure, the enclosure will break up, leading to cell lysis.

In some embodiments, the selectiveness of the lysing for specific cell type(s) depends on the gap size and the uniformity of the gap size; the more uniform the gap size, the more consistent is the lysing results.

As is well known, different cell types have different maximum and minimum natural dimensions. Herein the term “natural dimension” of a cell type refers to the average measurable size (in length) of a specific cell type that include either non-cultured cells in their natural in vivo conditions or cultured cells when they are suspended in a solution that mimics a state of physiological homeostasis. Depending on the shape and structure of different cell types, each cell type has a plurality of measurable dimensions. For example, mature human red blood cells (RBCs) in their natural state have a biconcave disc shape, with an average diameter of around 6-8 μm and average disc thickness of around 2 μm. The maximum natural dimension of the RBCs refers to the average diameter of the disc; the minimum natural dimension of the RBCs refers to the average disc thickness of the disc. In contrast, platelets in unactivated state are biconvex discoid (lens-shaped) structures and 2-3 μm in greatest diameter (maximum dimension), much smaller than the minimum natural dimension of the RBCs. WBCs, on the other hand, have the largest size as compared to RBCs and platelets, ranging from 7-30 μm in diameter, depending on the subtype.

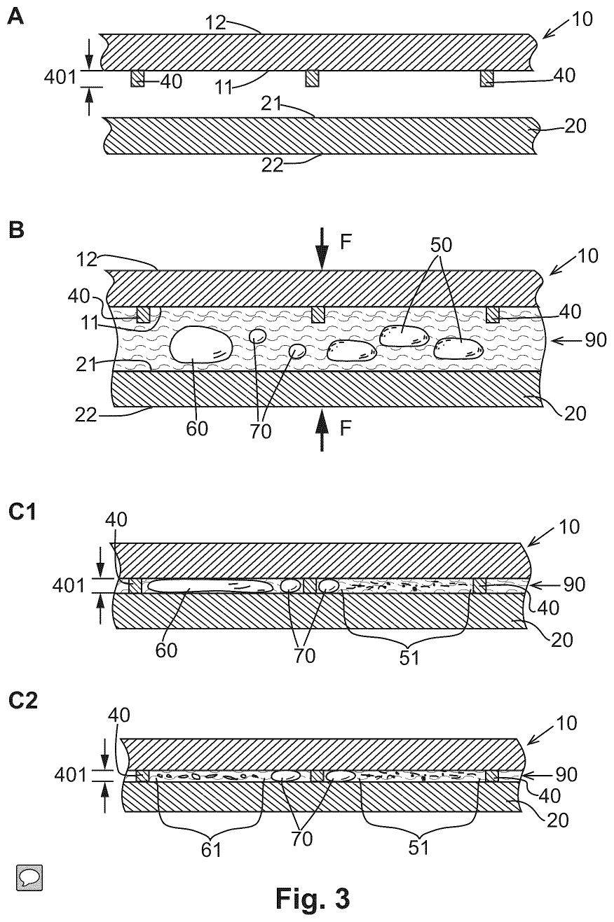

FIG. 3 shows exemplary embodiments of the device and method for platelet analysis as provided by the present invention, which mechanically lyse red blood cells and optionally white blood cells in a selective manner for improved viewing and imaging of platelet in blood sample. As shown in the figure, the device comprises a first plate 10, a second plate 20, and spacers 40. Both plates comprise, on the respective inner surface (11 and 21), a sample contact area (not indicated) for contacting blood sample. The spacers 40 are fixed to the inner surface of the first plate 11 and have a predetermined uniform height 401. It should be noted, however, in some embodiments, the spacers are fixed to the inner surface(s) of the second plate 20, or both the first plate 10 and the second plate 20. Panel (A) shows an open configuration of the device, in which, as discussed above, the first plate 10 and the second plate 20 are separated apart from each other, either partially or completely, and the spacing between the two plates is not regulated by the spacers 40.

Panel (B) of FIG. 3 shows that the two plates are used to spread a blood sample 90 that is deposited therebetween and contains platelets 70, red blood cells 50, and white blood cells 70. After the blood sample 90 (whole blood or partial blood sample, undiluted or diluted) is deposited on one or both of the plates at the open configuration, the two plates are brought to face each other with their inner surfaces 11 and 21, as shown in the figure. And a compressing force F is applied to the outer surfaces of the two plates 12 and 22 to force the two plates to enter the closed configuration. During this process, at least a part of the blood sample 90 is spread between the two plates while its thickness is reduced as the spacing between the two plates is decreased.

The natural dimensions of each cell type are critical factors in determining whether the cell type is susceptible to lysing by mechanical forces. Panels (C1) and (C2) of FIG. 3 show two exemplary embodiments of the device at the closed configuration after the compressing is completed, in which at least a part of the blood sample 90 is compressed by the two plates into a layer of uniform thickness, and in the layer a substantial fraction of platelets 70 remain unlysed while a substantial fraction of RBCs 60 or both RBC 60 and WBC 70 are selectively lysed by the mechanical pressure of the plates. As discussed above, when the spacing between the two plates is reduced to smaller than the minimum dimension of RBCs, the two plates compresses and deforms the RBCs in the uniform layer, leading to an increased internal pressure within RBCs' cell enclosure. When the internal pressure ramps up to exceed the tolerable threshold of RBCs' enclosure, the enclosure breaks up and releases the enclosed content, thus the cells are lysed. In some embodiments, at the closed configuration, the spacing between the two plates is regulated by the spacers. As exemplified in the figure, when the spacer height is selected to be smaller than the minimum dimension of the RBCs, but larger than the maximum dimension of the platelets, the compressing of the two plates to enter the closed configuration creates the mechanical pressure for the RBCs to be lysed, while leaving the majority of the platelets in the layer of uniform thickness spared.

Other factors affecting the selectiveness of the mechanical lysis include, but not limited to cell flexibility, cell membrane permeability, sample salt concentrations also play a role. For example, empirical evidence suggests that WBCs, particularly their cell membrane, exhibit much higher flexibility as compared to RBCs. Therefore, although normally larger in size than RBCs, WBCs are less susceptible to the mechanical force as compared to RBCs. Panel (C1) shows that a particular spacer height 401 is selected such that only RBCs 60 are lysed in the layer of uniform thickness, while platelets 70 and WBCs 60 remain unlysed although WBCs 60 are compressed and significantly deformed by the plates. Panel (C2) shows that a further smaller spacer height 401 as compared to panel (C1) is selected such that a substantial fraction of both RBCs 60 and WBCs 70 are lysed while a substantial fraction of platelets remains unlysed.

In some embodiments, RBCs are selectively lysed in the sample, and WBCs and platelets remain unlysed, and the spacer height is equal to or less than 2 um (micron or micrometer), 1.9 um, 1.8 um, 1.7 um, 1.6 um, 1.5 um, 1.4 um, 1.3 um, 1.2 um, 1.1 um, or 1.0 um, or in a range between any of the two values.

In some embodiments, both RBCs and WBCs are selectively lysed in the sample, and platelets remain unlysed, and the spacer height is equal to or less than 1.0 um, 0.9 um, 0.8 um, 0.7 um, 0.6 um, 0.5 um, 0.4 um, 0.3 um, or 0.2 um, or in a range between any of the two values.

In some embodiments, RBCs are selectively lysed in the sample, and platelets remain unlysed, and the spacer height is equal to or less than 2 μm, 1.9 um, 1.8 um, 1.7 um, 1.6 um, 1.5 um, 1.4 um, 1.3 um, 1.2 um, 1.1 um, 1.0 um, 0.9 um, 0.8 um, 0.7 um, 0.6 um, 0.5 um, 0.4 um, 0.3 um, or 0.2 um, or in a range between any of the two values.

### 2. Chemical Lysing

In some embodiments, chemical reagent(s) and/or biological reagent(s) is/are used to: facilitate 1) the selective lysing of the RBCs and/or WBCs in the sample; and/or 2) facilitate the protection of the platelets from lysing, for the better assessment of the platelets. These bio/chemical reagents are termed as “lysing agent” hereinafter.

In some embodiments, the lysing agent is preloaded into the sample before being analyzed in the QMAX device.

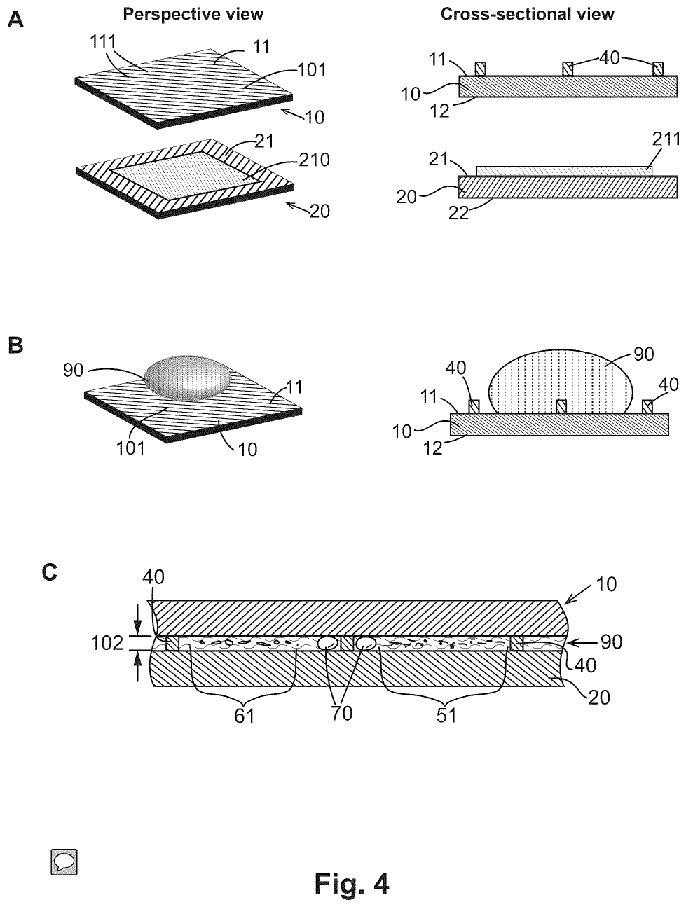

In some embodiments, the lysing agent is coated on the sample contact area of one or both of the plates. FIG. 4 shows an exemplary embodiment of the device and method for platelet analysis as provided by the present invention, which selectively lyse RBCs and WBCs using lysing agent stored on the plate(s). Panel (A) and (B) shows both perspective and cross-sectional views of the device at an open configuration. As shown in the figure, the device comprises a first plate 10, a second plate 20, and spacers 40. The spacers 40 are fixed to the first plate inner surface 11. Both plates comprise, on their respective inner surface (11 and 21), a sample contact area (not indicated) for contacting blood sample. Panel (A) shows that the second plate 20 comprises, on its sample contact area, a storage site 210 (not indicated in cross-sectional view), which contains a lysing reagent 211 (not shown in perspective view). The lysing reagent 211 is configured such that, upon contacting the blood sample, it is dissolved into the sample and diffuses therein, and the addition of the lysing agent 211 in the blood sample results in the selective lysis of RBCs and WBCs, while platelets remain unlysed. Panel (B) shows the deposition of a blood sample 90 on the sample contact area of the first plate 10. It should be noted, however, in some embodiments, the sample is deposited on the sample contact area(s) of the second plate 20, or both plates. Panel (C) shows the closed configuration of the device, in which: at least a part of the blood sample 90 is compressed by the two plates into a layer of uniform thickness, and inside the layer a substantial faction of platelets 70 remain unlysed while a substantial fraction of both RBC 60 and WBC 70 are selectively lysed as a result of the addition of the lysing agent 211 into the layer.

In some embodiments, the lysing agent includes, but not limited to, ammonium chloride, organic quaternary ammonium surfactants, cyanide salts, any other chemicals or biological reagent known to skilled artisan in the field, and any combination thereof.

In some embodiments, the lysing agent includes more than one species. In some embodiments, some species of the lysing agent are preloaded in the sample before being analyzed in the QMAX device, and some species of the lysing agent is coated on the QMAX device.

### 3. Combination

In some embodiments, both mechanical lysing and chemical lysing as discussed above are used to selectively lyse the RBCs and/or WBCs in the sample.

In some embodiments, the QMAX device comprises: 1) spacers that have a selected height; and 2) lysing agent on one or both the sample contact areas. The lysing agent facilitates: (a) the lysing of the targeted lysing component, and/or (b) the unlysing of non-targeted lysing components. The spacer height and the lysing agent are configured such that their combinatory effect results in the selective lysing of RBCs and optionally WBCs and the unlysing of the platelets in the layer of uniform thickness.

### Imaging

It is another aspect of the present invention to use imaging as the detection method to analyze the platelets confined in the sample layer between the two plates. In some embodiments, the present invention provides clear advantages for the imaging and analyzing of platelets after lysing the RBCs, which are abundant in whole blood sample and have much larger size, thereby may obscure the light path for the imaging.

In some embodiments, optical images are taken of the platelets under bright field illumination. For optical imaging, the platelets may be stained by colorant or not stained. In some embodiments, direct optical images are taken of the platelets without any colorant staining. In some embodiments, the platelets are stained by colorant pre-loaded into the blood sample before being analyzed by QMAX device and/or coated on one or both of the plates of the QMAX device. The term “colorant” as used herein refers to any reagent capable of causing a change in color in its target object that it becomes associated with. In some embodiments, the colorant is added to the sample to cause a differential staining of the platelets, rendering the platelets exhibit different color or color intensity than the surrounding substances (e.g. plasma, RBCs or RBCs residues). In some embodiments, the colorant is added to the sample to stain the platelets with no obvious differences from the surrounding substances.

In some embodiments, fluorescent images are taken of the platelets that are stained by fluorescently-labeled reagent. The fluorescently-labeled reagent is pre-loaded into the blood sample before being analyzed by QMAX device and/or coated on one or both of the plates of the QMAX device. Similar to the colorant as discussed above, in some embodiments, the fluorescently-labeled reagent differentially stains the platelets, for instance, it only stains the platelets, rendering only platelets in the sample emitting fluorescence upon stimulation, or it stains more substances besides platelets, but rendering the platelets emitting fluorescence with different parameters (e.g. excitation or emission spectra, intensity) than the surrounding substances. In some embodiments, the fluorescently-labeled reagent stains the platelets and other surrounding substances with no obvious difference. In some embodiments, the colorant is selected from the group consisting of: Acid fuchsin, Alcian blue 8 GX, Alizarin red S, Aniline blue WS, Auramine O, Azocarmine B, Azocarmine G, Azure A, Azure B, Azure C, Basic fuchsine, Bismarck brown Y, Brilliant cresyl blue, Brilliant green, Carmine, Chlorazol black E, Congo red, C.I. Cresyl violet, Crystal violet, Darrow red, Eosin B, Eosin Y, Erythrosin, Ethyl eosin, Ethyl green, Fast green F C F, Fluorescein Isothiocyanate, Giemsa Stain, Hematoxylin, Hematoxylin & Eosin, Indigo carmine, Janus green B, Jenner stain 1899, Light green SF, Malachite green, Martius yellow, Methyl orange, Methyl violet 2B, Methylene blue, Methylene blue, Methylene violet, (Bernthsen), Neutral red, Nigrosin, Nile blue A, Nuclear fast red, Oil Red, Orange G, Orange II, Orcein, Pararosaniline, Phloxin B, Protargol S, Pyronine B, Pyronine, Resazurin, Rose Bengal, Safranine O, Sudan black B, Sudan Ill, Sudan IV, Tetrachrome stain (MacNeal), Thionine, Toluidine blue, Weigert, Wright stain, and any combination thereof.

In some embodiments, the fluorescently-labeled reagent comprises fluorescent molecules (fluorophores), including, but not limited to, IRDye8000 W, Alexa 790, Dylight 800, fluorescein, fluorescein isothiocyanate, succinimidyl esters of carboxyfluorescein, succinimidyl esters of fluorescein, 5-isomer of fluorescein dichlorotriazine, caged carboxyfluorescein-alanine-carboxamide, Oregon Green 488, Oregon Green 514; Lucifer Yellow, acridine Orange, rhodamine, tetramethylrhodamine, Texas Red, propidium iodide, JC-1 (5,5′,6,6′-tetrachloro-1,1′,3,3′-tetraethylbenzimidazoylcarbocyanine iodide), tetrabromorhodamine 123, rhodamine 6G, TMRM (tetramethyl rhodamine methyl ester), TMRE (tetramethyl rhodamine ethyl ester), tetramethylrosamine, rhodamine B and 4-dimethylaminotetramethylrosamine, green fluorescent protein, blue-shifted green fluorescent protein, cyan-shifted green fluorescent protein, redshifted green fluorescent protein, yellow-shifted green fluorescent protein, 4-acetamido-4′-isothiocyanatostilbene-2,2′disulfonic acid; acridine and derivatives, such as acridine, acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-1-naphthyl)maleimide; anthranilamide; 4,4-difluoro-5-(2-thienyl)-4-bora-3a,4a diaza-5-indacene-3-propionic acid BODIPY; cascade blue; Brilliant Yellow; coumarin and derivatives: coumarin, 7-amino-4-methylcoumarin (AMC, Coumarin 120), 7-amino-4-trifluoromethylcoumarin (Coumarin 151); cyanine dyes; cyanosine; 4′,6-diaminidino-2-phenylindole (DAPI); 5′,5″-dibromopyrogallol sulfonaphthalein (Bromopyrogallol Red); 7-diethylamino-3-(4′-isothiocyanatophenyl)-4-methylcoumarin; diethylenetriaamine pentaacetate; 4,4′-diisothiocyanatodihydro-stilbene-2,2′-disulfonic acid; 4,4′-diisothiocyanatostilbene-2,2′-disulfonic acid; 5-(dimethylamino]naphthalene-1-sulfonyl chloride (DNS, dansylchloride); 4-dimethylaminophenylazophenyl-4′-isothiocyanate (DABITC); eosin and derivatives: eosin, eosin isothiocyanate, erythrosin and derivatives: erythrosin B, erythrosin, isothiocyanate; ethidium; fluorescein and derivatives: 5-carboxyfluorescein (FAM), 5-(4,6-dichlorotriazin-2-yl)amino- -fluorescein (DTAF), 2′,7′dimethoxy-4′5′-dichloro-6-carboxyfluorescein (JOE), fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; ophthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of 5 sulforhodamine (Texas Red); N,N,N′,N′-tetramethyl-6-carboxyrhodamine (TAMRA); tetramethyl rhodamine; tetramethyl hodamine isothiocyanate (TRITC); riboflavin; 5-(2′-aminoethyl)aminonaphthalene-1-sulfonic acid (EDANS), 4-(4′-dimethylaminophenylazo)benzoic acid (DABCYL), rosolic acid; CAL Fluor Orange 560; terbium chelate derivatives; Cy 3; Cy 5; Cy 5.5; Cy 7; IRD 700; IRD 800; La Jolla Blue; phthalo cyanine; and naphthalo cyanine, coumarins and related dyes, xanthene dyes such as rhodols, resorufins, bimanes, acridines, isoindoles, dansyl dyes, aminophthalic hydrazides such as luminol, and isoluminol derivatives, aminophthalimides, aminonaphthalimides, aminobenzofurans, aminoquinolines, dicyanohydroquinones, fluorescent europium and terbium complexes; combinations thereof, and the like. Suitable fluorescent proteins and chromogenic proteins include, but are not limited to, a green fluorescent protein (GFP), including, but not limited to, a GFP derived from Aequorea victoria or a derivative thereof, e.g., a “humanized” derivative such as Enhanced GFP; a GFP from another species such as Renilla reniformis, Renilla muller, or Ptilosarcus guernyi; “humanized” recombinant GFP (hrGFP); any of a variety of fluorescent and colored proteins from Anthozoan species; any combination thereof; and the like.

In some embodiments, fluorescently-labeled nucleic acid dyes are used to stain the platelets, which are capable of differentiating platelets from mature RBCs by highlighting the nuclei that exist in the former type of cells but not the latter. In some embodiments, these fluorescently-labeled nucleic acid dyes include, but not limited to, Acridine homodimer, Acridine orange, 7-AAD (7-amino-actinomycin D), Actinomycin D, ACMA, DAPI, Dihydroethidium, Ethidium bromide, Ethidium homodimer-1 (EthD-1), Ethidium homodimer-2 (EthD-2), Ethidium monoazide, Hexidium iodide, Hoechst 33258 (bis-benzimide), Hoechst 33342, Hoechst 34580, Hydroxystilbamidine, LDS 751, Nuclear yellow, Propidium iodide (PI); Quant-iT PicoGreen, Quant-iT OliGreen, SYBR Gold, SYBR Green I, SYBR Safe DNA stain, SYTOX Blue, SYTOX Green, SYTOX Orange, SYTOX Red, POPO-1, BOBO-1, YOYO-1, TOTO-1, JOJO-1, OPO-3, LOLO-1, BOBO-3, YOYO-3, TOTO-3, PO-PRO-1, YO-PRO-1, TO-PRO-1, JO-PRO-1, PO-PRO-3, YO-PRO-3, TO-PRO-3, TO-PRO-5, SYTO 40, SYTO 41, SYTO 42, SYTO 45, SYTO 81, SYTO 80, SYTO 82, SYTO 83, SYTO 84, SYTO 85, SYTO 64, SYTO 61, SYTO 17, SYTO 59, SYTO 62, SYTO 60, SYTO 63, and any combination thereof.

In some embodiments, both optical imaging and fluorescent imaging are used in combination for the detection and analysis of the platelets.

### System for Platelet Analysis

It is another aspect of the present invention to provide a system for platelet analysis that is easy-to-operate with improved viewing/counting of platelets in a very small volume of blood sample. In many embodiments, there is no need to dilute the sample or only need for slight dilution. And in certain embodiments, the system enables remote health monitoring, counseling, etc.

In some embodiments, the system comprises:
(a) a QMAX device as described in any foregoing or following embodiment;
(b) an imager, comprising a camera and a light source for imaging the platelets in the relevant volume of the sample; and
(c) a processor, comprising electronics, signal processors, hardware and software for receiving and processing the images and identifying and analyzing the platelets in the images.

In some embodiments, the system provides hardware and software for optical imaging as described above, including, but not limited to, a light source and optics providing bright-filed illumination of the sample in the QMAX device, imager and optics adapted for the imager to acquire optical images under bright-field illumination, and optionally software installed on the processor for processing of the optical images for the identification and analysis of the platelets in the images.

In some embodiments, the system provides hardware and software for fluorescent imaging as described above, including, but not limited to, a light source and optics (e.g. excitation filter) providing illumination at one or a range of wavelengths of the sample in the QMAX device, imager and optics (e.g. emission filter) adapted for the imager to acquire images at one or a range of wavelengths, and optionally software installed on the processor for processing of the fluorescent images for the identification and analysis of the platelets in the images.

In some embodiments, the mobile communication device, the light source, and the housing are configured to provide bright-field illumination of the sample, acquire and/or process optical images of the platelets in the relevant volume of the sample.

In some embodiments, the mobile communication device, the light source, and the housing are configured to provide fluorescent illumination of the sample, acquire and/or process fluorescent images of platelets that are fluorescently labeled in the relevant volume of the sample.

In some embodiments, a mobile communication device is utilized as the imager and optionally the image processor. In some embodiments, the system comprises:
(a) a QMAX device as described in any foregoing or following embodiment;
(b) a mobile communication device comprising:
   i. one or a plurality of cameras for imaging the platelets in the sample;
   ii. electronics, signal processors, hardware and software for receiving and/or processing the image of the platelets and for remote communication; and
(c) a light source from either the mobile communication device or an external source, wherein the light source is configured to provide illumination to the sample for imaging with the cameras.

In some embodiments, the system further comprises:
(d) a housing configured to hold the sample and to be mounted to the mobile communication device.

In some embodiments, the housing comprises optics for facilitating the imaging and/or signal processing of the sample by the mobile communication device, and a mount configured to hold the optics on the mobile communication device.

In some embodiments, the mobile communication device is configured to communicate test results to a medical professional, a medical facility or an insurance company.

In some embodiments, the mobile communication device is further configured to communicate information on the subject with the medical professional, medical facility or insurance company. In some embodiments, the mobile communication device is configured to receive a prescription, diagnosis or a recommendation from a medical professional. In some embodiments, the mobile communication device communicates with the remote location via a wifi or cellular network.

In some embodiments, the mobile communication device is a mobile phone.

### Examples of Present Invention

A1. A device for analyzing platelets in a blood sample, comprising:
   a first plate, a second plate, and spacers, wherein
      i. the plates are movable relative to each other into different configurations, including an open configuration and a closed configuration;
      ii. each of the plates has, on its respective sample surface, a sample contact area for contacting a blood sample, wherein the blood sample comprises red blood cells (RBCs) and platelets,
      iii. one or both of the plates comprise the spacers, and the spacers are fixed to the respective sample contact area, and
      iv. the height of the spacers is selected such that in the closed configuration, a substantial fraction of the RBCs in a relevant volume of the sample are lysed, and a substantial fraction of the platelets in the relevant volume of the sample are not lysed;
      wherein in the open configuration, the two plates are partially or entirely separated apart, the spacing between the plates is not regulated by the spacers, and the sample is deposited on one or both of the plates;
      wherein in the closed configuration, which is configured after deposition of the sample in the open configuration: the relevant volume of the sample is compressed by the two plates into a layer of highly uniform thickness, and the uniform thickness of the layer is confined by the sample contact surfaces of the plates and is regulated by the plates and the spacers; and
      wherein the relevant volume of the sample is a partial or entire volume of the sample.
AA1. A device for analyzing platelets in a blood sample, comprising:
   a first plate, a second plate, and spacers, wherein
      i. the plates are movable relative to each other into different configurations, including an open configuration and a closed configuration;
      ii. each of the plates has, on its respective sample surface, a sample contact area for contacting a blood sample, wherein the blood sample comprises red blood cells (RBCs) and platelets; and
      iii. one or both of the plates comprise the spacers, and the spacers are fixed to the respective plates; and
      iv. one or both of the plates comprise, on the respective sample contact area, a layer of lysing agent, wherein the lysing agent is configured such that, in the closed configuration, a substantial fraction of the RBCs in a relevant volume of the sample are lysed by the lysing agent dissolved in the relevant volume, and a substantial fraction of the platelets in the relevant volume of the sample are not lysed,
      wherein in the open configuration, the two plates are partially or entirely separated apart, the spacing between the plates is not regulated by the spacers, and the sample is deposited on one or both of the plates;
      wherein in the closed configuration, which is configured after deposition of the sample in the open configuration: the relevant volume of the sample is compressed by the two plates into a layer of highly uniform thickness, and the uniform thickness of the layer is confined by the sample contact surfaces of the plates and is regulated by the plates and the spacers; and
      wherein the relevant volume of the sample is a partial or entire volume of the sample.
B0. A system for analyzing platelets in a blood sample, comprising:
   (a) a device of embodiment A1 or AA1;
   (b) an imager, comprising a camera and a light source for imaging the platelets in the relevant volume of the sample; and
   (c) a processor, comprising electronics, signal processors, hardware and software for receiving and processing the images and identifying and analyzing the platelets in the images.
B1. A system for analyzing platelets in a blood sample, comprising:
   (a) a device of embodiment A1 or AA1;
   (b) a mobile communication device comprising:
      i. one or a plurality of cameras for imaging the platelets in the sample;
      ii. electronics, signal processors, hardware and software for receiving and/or processing the image of the platelets and for remote communication; and
   (c) a light source from either the mobile communication device or an external source, wherein the light source is configured to provide illumination to the sample for imaging with the cameras.
C1. A method of analyzing platelets in a blood sample, comprising the steps of:
   (a) obtaining a blood sample, which comprises red blood cells (RBCs) and platelets;
   (b) obtaining a first and second plates that are movable relative to each other into different configurations, including an open configuration and a closed configuration, wherein:
      i. each plate, on its respective surface, has a sample contact area for contacting the sample, and
      ii. one or both of the plates comprise spacers that are fixed with a respective sample contact surface,
         wherein the spacers have a predetermined substantially uniform height, and at least one of the spacers is inside the sample contact area;
   (c) depositing the sample on one or both of the plates when the plates are in an open configuration,
      wherein in the open configuration the two plates are partially or entirely separated apart and the spacing between the plates is not regulated by the spacers;
   (d) after (c), bringing the two plates together and pressing the plates into a closed configuration;
   (e) while the plates are at the closed configuration, acquiring images of the platelets in a relevant volume of the sample; and
   (f) identifying and analyzing the platelets in the acquired images,
      wherein in the closed configuration: the relevant volume of the sample is compressed by the two plates into a layer of highly uniform thickness, the uniform thickness of the layer is confined by the sample surfaces of the two plates and is regulated by the spacers and the plates,
      wherein the height of the spacers is selected such that in the closed configuration, a substantial fraction of the RBCs of the sample in the relevant volume of the sample are lysed, and a substantial fraction of the platelets in the relevant volume of the sample are not lysed; and
      wherein the relevant volume of the sample is a partial or entire volume of the sample.
CC1. A method of analyzing platelets in a blood sample, comprising the steps of:
   (a) obtaining a blood sample, which comprises red blood cells (RBCs) and platelets;
   (b) obtaining a first and second plates that are movable relative to each other into different configurations, including an open configuration and a closed configuration, wherein:
      i. each plate, on its respective surface, has a sample contact area for contacting the sample,
      ii. one or both of the plates comprise spacers that are fixed with a respective sample contact area, and
      iii. one or both of the plates comprise, on the respective sample contact area, a layer of lysing agent, wherein the lysing agent is configured such that, in the closed configuration, a substantial fraction of the RBCs in a relevant volume of the sample are lysed by the lysing agent that is dissolved in the relevant volume, and a substantial fraction of the platelets in the relevant volume of the sample are not lysed,
         wherein the spacers have a predetermined substantially uniform height, and at least one of the spacers is inside the sample contact area;
   (c) depositing the sample on one or both of the plates when the plates are in an open configuration,
      wherein in the open configuration the two plates are partially or entirely separated apart and the spacing between the plates is not regulated by the spacers;
   (d) after (c), bringing the two plates together and pressing the plates into a closed configuration;
   (e) while the plates are at the closed configuration, acquiring images of the platelets in the relevant volume of the sample; and
   (f) identifying and analyzing the platelets in the acquired images,
      wherein in the closed configuration: the relevant volume of the sample is compressed by the two plates into a layer of highly uniform thickness, the uniform thickness of the layer is confined by the sample surfaces of the two plates and is regulated by the spacers and the plates, and
      wherein the relevant volume of the sample is a partial or entire volume of the sample.
A3. The device, system, or method of any prior embodiments, wherein at least one of the plates is transparent.
A4. The device, system, or method of any prior embodiments, wherein one or both of the plates comprise, on the respective sample contact area, a dye that, upon contacting the sample, is dissolved in the sample and stains the platelets.
A5. The device, system, or method of embodiment A4, wherein the dye is fluorescently labeled.
A6. The device, system, or method of embodiment A4, wherein the dye is acridine orange (AO).
A7. The device, system, or method of any prior embodiments, wherein the blood sample is stained before being analyzed.
A8. The device, system, or method of any prior embodiments, wherein on one or both the sample contact areas, the respective plate further comprises a layer of a reagent.
A9. The device, system, or method of embodiment A15, wherein the reagent facilitates: (a) the lysing of the RBCs and/or WBCs, and/or (b) the unlysing of platelets.
A10. The device, system, or method of embodiment A15, wherein the reagent is used for bio/chemical assay of the platelets.
A11. The device, system, or method of any prior embodiment, wherein the lysing agent is selected from the group consisting of: ammonium chloride, organic quaternary ammonium surfactants, cyanide salts, and any combination thereof.
A12. The device, system, or method of any prior embodiments, wherein the substantial fraction is at least 51%, 60%, 70%, 80%, 90%, 95% or 99% of a component in the relevant volume of the sample.
A13. The device, system, or method of any prior embodiments, wherein the thickness variation of the layer of highly uniform thickness over the lateral area of the relevant volume is equal to or less than 40%, 30%, 20%, 15%, 10%, 7%, 5%, 3%, or 1%, or in a range between any of the two values, wherein the thickness variation is relative to the average thickness of the lateral area.
A14. The device, system, or method of any prior embodiments, wherein the area of the highly uniform layer is equal to or larger than 0.1 mm², 0.5 mm², 1 mm², 3 mm², 5 mm², 10 mm², 20 mm², 50 mm², 70 mm², 100 mm², 200 mm², 500 mm², 800 mm², 1000 mm², 2000 mm², 5000 mm², 10000 mm², 20000 mm², 50000 mm², or 100000 mm²; or in a range between any of the two values.
A15. The device, system, or method of any prior embodiments, wherein the blood sample is diluted or undiluted whole blood.
A16. The device, system, or method of any prior embodiments, wherein the blood sample is partial blood sample.
A17. The device, system, or method of any prior embodiments, wherein the spacer height is equal to or less than 2 um, 1.9 um, 1.8 um, 1.7 um, 1.6 um, 1.5 um, 1.4 um, 1.3 um, 1.2 um, 1.1 um, 1.0 um, 0.9 um, 0.8 um, 0.7 um, 0.6 um, 0.5 um, 0.4 um, 0.3 um, or 0.2 um, or in a range between any of the two values.
A18. The device, system, or method of any prior embodiments, wherein in the closed configuration, a substantial fraction of white blood cells (WBCs) in the relevant volume of the sample are lysed, and the spacer height is equal to or less than 1.0 um, 0.9 um, 0.8 um, 0.7 um, 0.6 um, 0.5 um, 0.4 um, 0.3 um, or 0.2 um, or in a range between any of the two values.
B2. The system of any prior embodiments, further comprising:
(d) a housing configured to hold the sample and to be mounted to the mobile communication device.
B3. The system of any prior embodiments, wherein the mobile communication device, the light source, and the housing are configured to provide bright-field illumination of the sample, acquire and/or process optical images of the platelets in the relevant volume of the sample.
B4. The system of any prior embodiments, wherein the mobile communication device, the light source, and the housing are configured to provide fluorescent illumination of the sample, acquire and/or process fluorescent images of platelets that are fluorescently labeled in the relevant volume of the sample.
B5. The system of any prior embodiments, wherein the housing comprises optics for facilitating the imaging and/or signal processing of the sample by the mobile communication device, and a mount configured to hold the optics on the mobile communication device.
B6. The system of any of any prior embodiments, wherein the mobile communication device is configured to communicate test results to a medical professional, a medical facility or an insurance company.
B7. The system of any prior embodiments, wherein the mobile communication device is further configured to communicate information on the subject with the medical professional, medical facility or insurance company.
B8. The system of any prior embodiments, wherein the mobile communication device is configured to receive a prescription, diagnosis or a recommendation from a medical professional.
B9. The system of any prior embodiments, wherein the mobile communication device communicates with the remote location via a wifi or cellular network.
B10. The system of any prior embodiments, wherein the mobile communication device is a mobile phone.
C2. The method of any prior embodiments, wherein the step (e) of acquiring the images is performed by a mobile communication device that comprises:
   i. one or a plurality of cameras for imaging the platelets in the sample;
   ii. electronics, signal processors, hardware and software for receiving and/or processing the image of the platelets and for remote communication; and
a light source from either the mobile communication device or an external source.
C3. The method of any prior embodiments, wherein the step (e) of acquiring the images comprises:
i. acquiring optical images of the platelets in the relevant volume of the sample; and/or
   ii. acquiring fluorescent images of fluorescently-labeled platelets in the relevant volume of the sample in fluorescence mode, wherein the platelets are fluorescently labeled by a fluorescent dye that is pre-loaded into the sample or coated on the sample contact area of one or both of the plates.
C4. The method of any prior embodiments, wherein the step (f) of identifying and analyzing is performed by a mobile communication device that is configured to receive and/or process the image of the platelets.
C5. The method of any prior embodiments, wherein the analyzing comprises counting the number of the platelets in a first area of the images.
C6. The method of embodiment C5, wherein the analyzing further comprises calculating the concentration of platelet in the sample by:
   (1) determining the volume of the sample covered by the first area through timing the first area by the uniform height of the spacers; and
   (2) dividing the count number of the platelets in the first area by the volume determined in step (1).
E1. The device, system, or method of any prior embodiments, wherein the spacers have:
   i. a shape of pillar with substantially uniform cross-section and a flat top surface;
   ii. a ratio of the width to the height equal or larger than one;
   iii. a filling factor of equal to 1% or larger; and
   iv. a product of the filling factor and the Young's modulus of the spacer is 2 MPa or larger,
   wherein the filling factor is the ratio of the spacer contact area to the total plate area.
E2. The device, system, or method of any prior embodiments, wherein an average value of the uniform thickness of the layer is substantially the same as the uniform height of the spacer with a variation of less than 10%.
E4. The device, system, or method of any prior embodiments, wherein in the closed configuration at least 90% of the RBCs are lysed and at least 90% of the platelets are not lysed.
E5. The device, system, or method of any prior embodiments, wherein in the closed configuration at least 99% of the RBCs are lysed and at least 99% of the platelets are not lysed.
E6. The device, system, or method of any prior embodiments, wherein the variation of the layer of uniform thickness is less than 30 nm.
E7. The device, system, or method of any prior embodiments, wherein the layer of uniform thickness sample has a thickness uniformity of up to +/−5%.
E8. The device, system, or method of any prior embodiments, wherein the spacers are pillars with a cross-sectional shape selected from round, polygonal, circular, square, rectangular, oval, elliptical, or any combination of the same.
E9. The device, system, or method of any prior embodiments, wherein the spacers have:
   i. a shape of pillar with substantially uniform cross-section and a flat top surface;
   ii. a ratio of the width to the height equal or larger than one;
   iii. a predetermined constant inter-spacer distance that is in the range of 10 μm to 200 μm;
   iv. a filling factor of equal to 1% or larger; and
   v. a product of the filling factor and the Young's modulus of the spacer is 2 MPa or larger.
   wherein the filling factor is the ratio of the spacer contact area to a total plate area.
E10. The device, system, or method of any prior embodiments, wherein pressing the plates into the closed configuration is conducted either in parallel or sequentially, the parallel pressing applies an external force on an intended area at the same time, and the sequential pressing applies an external force on a part of an intended area and gradually move to other area.
E11. The device, system, or method of any prior embodiments, wherein the blood sample is analyzed by:
   i. illuminating at least part of the blood sample in the layer of uniform thickness;
   ii. obtaining one or more images of the cells using a CCD or CMOS sensor;
   iii. identifying the platelets in the image using a computer; and
   iv. counting a number of platelets in an area of the image.
E12. The device, system, or method of any prior embodiments, wherein the layer of uniform thickness sample has a thickness uniformity of up to +/−5%.

### Related Documents

The present invention includes a variety of embodiments, which can be combined in multiple ways as long as the various components do not contradict one another. The embodiments should be regarded as a single invention file: each filing has other filing as the references and is also referenced in its entirety and for all purpose, rather than as a discrete independent. These embodiments include not only the disclosures in the current file, but also the documents that are herein referenced, incorporated, or to which priority is claimed.

### (1) Definitions

The terms used in describing the devices, systems, and methods herein disclosed are defined in the current application, or in PCT Application (designating U.S.) Nos. PCT/US2016/045437 and PCT/US0216/051775, which were respectively filed on Aug. 10, 2016 and Sep. 14, 2016, and U.S. Provisional Application No. 62/456,065, which was filed on Feb. 7, 2017, and are all hereby incorporated in reference by their entireties.

### (2) Spacer Ad Uniformity

The devices, systems, and methods herein disclosed can include or use QMAX cards for sample detection, analysis, and quantification. In some embodiments, the QMAX card comprises spacers, which help to render at least part of the sample into a layer of high uniformity. The structure, material, function, variation and dimension of the spacers, as well as the uniformity of the spacers and the sample layer, are herein disclosed, or listed, described, and summarized in PCT Application (designating U.S.) Nos. PCT/US2016/045437 and PCT/US0216/051775, which were respectively filed on Aug. 10, 2016 and Sep. 14, 2016, and U.S. Provisional Application No. 62/456,065, which was filed on Feb. 7, 2017, and are all hereby incorporated by reference in their entireties.

### (3) Hinges, Notches, Recesses and Sliders

The devices, systems, and methods herein disclosed can include or use QMAX cards for sample detection, analysis, and quantification. In some embodiments, the QMAX card comprises hinges, notches, recesses, and sliders, which help to facilitate the manipulation of the QMAX card and the measurement of the samples. The structure, material, function, variation and dimension of the hinges, notches, recesses, and sliders are herein disclosed, or listed, described, and summarized in PCT Application (designating U.S.) Nos. PCT/US2016/045437 and PCT/US0216/051775, which were respectively filed on Aug. 10, 2016 and Sep. 14, 2016, and U.S. Provisional Application No. 62/456,065, which was filed on Feb. 7, 2017, and are all hereby incorporated by reference in their entireties.

### (4) Card, Sliders, and Smartphone Detection System

The devices, systems, and methods herein disclosed can include or use QMAX cards for sample detection, analysis, and quantification. In some embodiments, the QMAX cards are used together with sliders that allow the card to be read by a smartphone detection system. The structure, material, function, variation, dimension and connection of the QMAX card, the sliders, and the smartphone detection system are herein disclosed, or listed, described, and summarized in PCT Application (designating U.S.) Nos. PCT/US2016/045437 and PCT/US0216/051775, which were respectively filed on Aug. 10, 2016 and Sep. 14, 2016, and U.S. Provisional Application No. 62/456,065, which was filed on Feb. 7, 2017, and are all hereby incorporated by reference in their entireties.

### (5) Detection Methods

The devices, systems, and methods herein disclosed can include or be used in various types of detection methods. The detection methods are herein disclosed, or listed, described, and summarized in PCT Application (designating U.S.) Nos. PCT/US2016/045437 and PCT/US0216/051775, which were respectively filed on Aug. 10, 2016 and Sep. 14, 2016, and U.S. Provisional Application No. 62/456,065, which was filed on Feb. 7, 2017, and are all hereby incorporated by reference in their entireties.

### (6) Labels

The devices, systems, and methods herein disclosed can employ various types of labels. The labels are herein disclosed, or listed, described, and summarized in PCT Application (designating U.S.) Nos. PCT/US2016/045437 and PCT/US0216/051775, which were respectively filed on Aug. 10, 2016 and Sep. 14, 2016, and U.S. Provisional Application No. 62/456,065, which was filed on Feb. 7, 2017, and are all hereby incorporated by reference in their entireties.

### (7) Biomarkers

The devices, systems, and methods herein disclosed can employ various types of biomarkers. The biomarkers are herein disclosed, or listed, described, and summarized in PCT Application (designating U.S.) Nos. PCT/US2016/045437 and PCT/US0216/051775, which were respectively filed on Aug. 10, 2016 and Sep. 14, 2016, and U.S. Provisional Application No. 62/456,065, which was filed on Feb. 7, 2017, and are all hereby incorporated by reference in their entireties.

### (8) Cloud

The devices, systems, and methods herein disclosed can employ cloud technology for data transfer, storage, and/or analysis. The related cloud technologies are herein disclosed, or listed, described, and summarized in PCT Application (designating U.S.) Nos. PCT/US2016/045437 and PCT/US0216/051775, which were respectively filed on Aug. 10, 2016 and Sep. 14, 2016, and U.S. Provisional Application No. 62/456,065, which was filed on Feb. 7, 2017, and are all hereby incorporated by reference in their entireties.

### (9) Applications (Field and Samples)

The devices, systems, and methods herein disclosed can be used for various applications (fields and samples). The applications are herein disclosed, or listed, described, and summarized in PCT Application (designating U.S.) Nos. PCT/US2016/045437 and PCT/US0216/051775, which were respectively filed on Aug. 10, 2016 and Sep. 14, 2016, and U.S. Provisional Application No. 62/456,065, which was filed on Feb. 7, 2017, and are all hereby incorporated by reference in their entireties.

### Additional Notes

Further examples of inventive subject matter according to the present disclosure are described in the following enumerated paragraphs.

It must be noted that as used herein and in the appended claims, the singular forms “a”, “an”, and “the” include plural referents unless the context clearly dictates otherwise, e.g., when the word “single” is used. For example, reference to “an analyte” includes a single analyte and multiple analytes, reference to “a capture agent” includes a single capture agent and multiple capture agents, reference to “a detection agent” includes a single detection agent and multiple detection agents, and reference to “an agent” includes a single agent and multiple agents.

As used here, the term “analyte” refers to a molecule (e.g., a protein, peptides, DNA, RNA, nucleic acid, or other molecule) or molecules, cells, tissues, viruses, and nanoparticles with different shapes. It can also be referred to as any substance that is suitable for testing in the present invention.

As used herein, the terms “adapted” and “configured” mean that the element, component, or other subject matter is designed and/or intended to perform a given function. Thus, the use of the terms “adapted” and “configured” should not be construed to mean that a given element, component, or other subject matter is simply “capable of” performing a given function. Similarly, subject matter that is recited as being configured to perform a particular function may additionally or alternatively be described as being operative to perform that function.

As used herein, the phrase, “for example,” the phrase, “as an example,” and/or simply the terms “example” and “exemplary” when used with reference to one or more components, features, details, structures, embodiments, and/or methods according to the present disclosure, are intended to convey that the described component, feature, detail, structure, embodiment, and/or method is an illustrative, non-exclusive example of components, features, details, structures, embodiments, and/or methods according to the present disclosure. Thus, the described component, feature, detail, structure, embodiment, and/or method is not intended to be limiting, required, or exclusive/exhaustive; and other components, features, details, structures, embodiments, and/or methods, including structurally and/or functionally similar and/or equivalent components, features, details, structures, embodiments, and/or methods, are also within the scope of the present disclosure.

As used herein, the phrases “at least one of” and “one or more of,” in reference to a list of more than one entity, means any one or more of the entity in the list of entity, and is not limited to at least one of each and every entity specifically listed within the list of entity. For example, “at least one of A and B” (or, equivalently, “at least one of A or B,” or, equivalently, “at least one of A and/or B”) may refer to A alone, B alone, or the combination of A and B.

As used herein, the term “and/or” placed between a first entity and a second entity means one of (1) the first entity, (2) the second entity, and (3) the first entity and the second entity. Multiple entity listed with “and/or” should be construed in the same manner, i.e., “one or more” of the entity so conjoined. Other entity may optionally be present other than the entity specifically identified by the “and/or” clause, whether related or unrelated to those entities specifically identified.

Where numerical ranges are mentioned herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, unless otherwise indicated or otherwise evident from the context and understanding of one of ordinary skill in the art.

In the event that any patents, patent applications, or other references are incorporated by reference herein and (1) define a term in a manner that is inconsistent with and/or (2) are otherwise inconsistent with, either the non-incorporated portion of the present disclosure or any of the other incorporated references, the non-incorporated portion of the present disclosure shall control, and the term or incorporated disclosure therein shall only control with respect to the reference in which the term is defined and/or the incorporated disclosure was present originally.

### 1. Samples

The devices, apparatus, systems, and methods herein disclosed can be used for samples such as but not limited to diagnostic samples, clinical samples, environmental samples and foodstuff samples. The types of sample include but are not limited to the samples listed, described and/or summarized in PCT Application (designating U.S.) Nos. PCT/US2016/045437 and PCT/US0216/051775, which were respectively filed on Aug. 10, 2016 and Sep. 14, 2016, and are hereby incorporated by reference by their entireties.

For example, in some embodiments, the devices, apparatus, systems, and methods herein disclosed are used for a sample that includes cells, tissues, bodily fluids and/or a mixture thereof. In some embodiments, the sample comprises a human body fluid. In some embodiments, the sample comprises at least one of cells, tissues, bodily fluids, stool, amniotic fluid, aqueous humour, vitreous humour, blood, whole blood, fractionated blood, plasma, serum, breast milk, cerebrospinal fluid, cerumen, chyle, chime, endolymph, perilymph, feces, gastric acid, gastric juice, lymph, mucus, nasal drainage, phlegm, pericardial fluid, peritoneal fluid, pleural fluid, pus, rheum, saliva, sebum, semen, sputum, sweat, synovial fluid, tears, vomit, urine, and exhaled breath condensate.

In some embodiments, the devices, apparatus, systems, and methods herein disclosed are used for an environmental sample that is obtained from any suitable source, such as but not limited to: river, lake, pond, ocean, glaciers, icebergs, rain, snow, sewage, reservoirs, tap water, drinking water, etc.; solid samples from soil, compost, sand, rocks, concrete, wood, brick, sewage, etc.; and gaseous samples from the air, underwater heat vents, industrial exhaust, vehicular exhaust, etc. In certain embodiments, the environmental sample is fresh from the source; in certain embodiments, the environmental sample is processed. For example, samples that are not in liquid form are converted to liquid form before the subject devices, apparatus, systems, and methods are applied.

In some embodiments, the devices, apparatus, systems, and methods herein disclosed are used for a foodstuff sample, which is suitable or has the potential to become suitable for animal consumption, e.g., human consumption. In some embodiments, a foodstuff sample includes raw ingredients, cooked or processed food, plant and animal sources of food, preprocessed food as well as partially or fully processed food, etc. In certain embodiments, samples that are not in liquid form are converted to liquid form before the subject devices, apparatus, systems, and methods are applied.

The subject devices, apparatus, systems, and methods can be used to analyze any volume of the sample. Examples of the volumes include, but are not limited to, about 10 mL or less, 5 mL or less, 3 mL or less, 1 microliter (μL, also “uL” herein) or less, 500 μL or less, 300 μL or less, 250 μL or less, 200 μL or less, 170 μL or less, 150 μL or less, 125 μL or less, 100 μL or less, 75 μL or less, 50 μL or less, 25 μL or less, 20 μL or less, 15 μL or less, 10 μL or less, 5 μL or less, 3 μL or less, 1 μL or less, 0.5 μL or less, 0.1 μL or less, 0.05 μL or less, 0.001 μL or less, 0.0005 μL or less, 0.0001 μL or less, 10 μL or less, 1 μL or less, or a range between any two of the values.

In some embodiments, the volume of the sample includes, but is not limited to, about 100 μL or less, 75 μL or less, 50 μL or less, 25 μL or less, 20 μL or less, 15 μL or less, 10 μL or less, 5 μL or less, 3 μL or less, 1 μL or less, 0.5 μL or less, 0.1 μL or less, 0.05 μL or less, 0.001 μL or less, 0.0005 μL or less, 0.0001 μL or less, 10 μL or less, 1 μL or less, or a range between any two of the values. In some embodiments, the volume of the sample includes, but is not limited to about 10 μL or less, 5 μL or less, 3 μL or less, 1 μL or less, 0.5 μL or less, 0.1 μL or less, 0.05 μL or less, 0.001 μL or less, 0.0005 μL or less, 0.0001 μL or less, 10 μL or less, 1 μL or less, or a range between any two of the values.

In some embodiments, the amount of the sample is about a drop of liquid. In certain embodiments, the amount of sample is the amount collected from a pricked finger or fingerstick. In certain embodiments, the amount of sample is the amount collected from a microneedle, micropipette or a venous draw.

In certain embodiments, the sample holder is configured to hold a fluidic sample. In certain embodiments, the sample holder is configured to compress at least part of the fluidic sample into a thin layer. In certain embodiments, the sample holder comprises structures that are configured to heat and/or cool the sample. In certain embodiments, the heating source provides electromagnetic waves that can be absorbed by certain structures in the sample holder to change the temperature of the sample. In certain embodiments, the signal sensor is configured to detect and/or measure a signal from the sample. In certain embodiments, the signal sensor is configured to detect and/or measure an analyte in the sample. In certain embodiments, the heat sink is configured to absorb heat from the sample holder and/or the heating source. In certain embodiments, the heat sink comprises a chamber that at least partly enclose the sample holder.

### 2. Applications

The devices, apparatus, systems, and methods herein disclosed can be used in various types of biological/chemical sampling, sensing, assays and applications, which include the applications listed, described and/or summarized in PCT Application (designating U.S.) No. PCT/US2016/045437, which was filed on Aug. 10, 2016, and is hereby incorporated by reference by its entirety.

In some embodiments, the devices, apparatus, systems, and methods herein disclosed are used in a variety of different application in various field, wherein determination of the presence or absence, quantification, and/or amplification of one or more analytes in a sample are desired. For example, in certain embodiments the subject devices, apparatus, systems, and methods are used in the detection of proteins, peptides, nucleic acids, synthetic compounds, inorganic compounds, organic compounds, bacteria, virus, cells, tissues, nanoparticles, and other molecules, compounds, mixtures and substances thereof. The various fields in which the subject devices, apparatus, systems, and methods can be used include, but are not limited to: diagnostics, management, and/or prevention of human diseases and conditions, diagnostics, management, and/or prevention of veterinary diseases and conditions, diagnostics, management, and/or prevention of plant diseases and conditions, agricultural uses, veterinary uses, food testing, environments testing and decontamination, drug testing and prevention, and others.

The applications of the present invention include, but are not limited to: (a) the detection, purification, quantification, and/or amplification of chemical compounds or biomolecules that correlates with certain diseases, or certain stages of the diseases, e.g., infectious and parasitic disease, injuries, cardiovascular disease, cancer, mental disorders, neuropsychiatric disorders and organic diseases, e.g., pulmonary diseases, renal diseases, (b) the detection, purification, quantification, and/or amplification of cells and/or microorganism, e.g., virus, fungus and bacteria from the environment, e.g., water, soil, or biological samples, e.g., tissues, bodily fluids, (c) the detection, quantification of chemical compounds or biological samples that pose hazard to food safety, human health, or national security, e.g. toxic waste, anthrax, (d) the detection and quantification of vital parameters in medical or physiological monitor, e.g., glucose, blood oxygen level, total blood count, (e) the detection and quantification of specific DNA or RNA from biological samples, e.g., cells, viruses, bodily fluids, (f) the sequencing and comparing of genetic sequences in DNA in the chromosomes and mitochondria for genome analysis or (g) the detection and quantification of reaction products, e.g., during synthesis or purification of pharmaceuticals.

In some embodiments, the subject devices, apparatus, systems, and methods are used in the detection of nucleic acids, proteins, or other molecules or compounds in a sample. In certain embodiments, the devices, apparatus, systems, and methods are used in the rapid, clinical detection and/or quantification of one or more, two or more, or three or more disease biomarkers in a biological sample, e.g., as being employed in the diagnosis, prevention, and/or management of a disease condition in a subject. In certain embodiments, the devices, apparatus, systems, and methods are used in the detection and/or quantification of one or more, two or more, or three or more environmental markers in an environmental sample, e.g. sample obtained from a river, ocean, lake, rain, snow, sewage, sewage processing runoff, agricultural runoff, industrial runoff, tap water or drinking water. In certain embodiments, the devices, apparatus, systems, and methods are used in the detection and/or quantification of one or more, two or more, or three or more foodstuff marks from a food sample obtained from tap water, drinking water, prepared food, processed food or raw food.

In some embodiments, the subject device is part of a microfluidic device. In some embodiments, the subject devices, apparatus, systems, and methods are used to detect a fluorescence or luminescence signal. In some embodiments, the subject devices, apparatus, systems, and methods include, or are used together with, a communication device, such as but not limited to: mobile phones, tablet computers and laptop computers. In some embodiments, the subject devices, apparatus, systems, and methods include, or are used together with, an identifier, such as but not limited to an optical barcode, a radio frequency ID tag, or combinations thereof.

In some embodiments, the sample is a diagnostic sample obtained from a subject, the analyte is a biomarker, and the measured amount of the analyte in the sample is diagnostic of a disease or a condition. In some embodiments, the subject devices, systems and methods further include receiving or providing to the subject a report that indicates the measured amount of the biomarker and a range of measured values for the biomarker in an individual free of or at low risk of having the disease or condition, wherein the measured amount of the biomarker relative to the range of measured values is diagnostic of a disease or condition.

In some embodiments, the sample is an environmental sample, and wherein the analyte is an environmental marker. In some embodiments, the subject devices, systems and methods includes receiving or providing a report that indicates the safety or harmfulness for a subject to be exposed to the environment from which the sample was obtained. In some embodiments, the subject devices, systems and methods include sending data containing the measured amount of the environmental marker to a remote location and receiving a report that indicates the safety or harmfulness for a subject to be exposed to the environment from which the sample was obtained.

In some embodiments, the sample is a foodstuff sample, wherein the analyte is a foodstuff marker, and wherein the amount of the foodstuff marker in the sample correlate with safety of the foodstuff for consumption. In some embodiments, the subject devices, systems and methods include receiving or providing a report that indicates the safety or harmfulness for a subject to consume the foodstuff from which the sample is obtained. In some embodiments, the subject devices, systems and methods include sending data containing the measured amount of the foodstuff marker to a remote location and receiving a report that indicates the safety or harmfulness for a subject to consume the foodstuff from which the sample is obtained.

### 3. Analytes, Biomarkers, and Diseases

The devices, apparatus, systems, and methods herein disclosed can be used for the detection, purification and/or quantification of various analytes. In some embodiments, the analytes are biomarkers that associated with various diseases. In some embodiments, the analytes and/or biomarkers are indicative of the presence, severity, and/or stage of the diseases. The analytes, biomarkers, and/or diseases that can be detected and/or measured with the devices, apparatus, systems, and/or method of the present invention include the analytes, biomarkers, and/or diseases listed, described and/or summarized in PCT Application (designating U.S.) Nos. PCT/US2016/045437 filed on Aug. 10, 2016, and PCT Application No. PCT/US2016/054025 filed on Sep. 27, 2016, and U.S. Provisional Application Nos. 62/234,538 filed on Sep. 29, 2015, 62/233,885 filed on Sep. 28, 2015, 62/293,188 filed on Feb. 9, 2016, and 62/305,123 filed on Mar. 8, 2016, which are all hereby incorporated by reference by their entireties. For example, the devices, apparatus, systems, and methods herein disclosed can be used in (a) the detection, purification and quantification of chemical compounds or biomolecules that correlates with the stage of certain diseases, e.g., infectious and parasitic disease, injuries, cardiovascular disease, cancer, mental disorders, neuropsychiatric disorders and organic diseases, e.g., pulmonary diseases, renal diseases, (b) the detection, purification and quantification of microorganism, e.g., virus, fungus and bacteria from environment, e.g., water, soil, or biological samples, e.g., tissues, bodily fluids, (c) the detection, quantification of chemical compounds or biological samples that pose hazard to food safety or national security, e.g. toxic waste, anthrax, (d) quantification of vital parameters in medical or physiological monitor, e.g., glucose, blood oxygen level, total blood count, (e) the detection and quantification of specific DNA or RNA from biosamples, e.g., cells, viruses, bodily fluids, (f) the sequencing and comparing of genetic sequences in DNA in the chromosomes and mitochondria for genome analysis or (g) to detect reaction products, e.g., during synthesis or purification of pharmaceuticals.

In some embodiments, the analyte can be a biomarker, an environmental marker, or a foodstuff marker. The sample in some instances is a liquid sample, and can be a diagnostic sample (such as saliva, serum, blood, sputum, urine, sweat, lacrima, semen, or mucus); an environmental sample obtained from a river, ocean, lake, rain, snow, sewage, sewage processing runoff, agricultural runoff, industrial runoff, tap water or drinking water; or a foodstuff sample obtained from tap water, drinking water, prepared food, processed food or raw food.

In any embodiment, the sample can be a diagnostic sample obtained from a subject, the analyte can be a biomarker, and the measured the amount of the analyte in the sample can be diagnostic of a disease or a condition.

In any embodiment, the devices, apparatus, systems, and methods in the present invention can further include diagnosing the subject based on information including the measured amount of the biomarker in the sample. In some cases, the diagnosing step includes sending data containing the measured amount of the biomarker to a remote location and receiving a diagnosis based on information including the measurement from the remote location.

In any embodiment, the biomarker can be selected from Tables B1, 2, 3 or 7 as disclosed in U.S. Provisional Application Nos. 62/234,538, 62/293,188, and/or 62/305,123, and/or PCT Application No. PCT/US2016/054,025, which are all incorporated in their entireties for all purposes. In some instances, the biomarker is a protein selected from Tables B1, 2, or 3. In some instances, the biomarker is a nucleic acid selected from Tables B2, 3 or 7. In some instances, the biomarker is an infectious agent-derived biomarker selected from Table B2. In some instances, the biomarker is a microRNA (miRNA) selected from Table B7.

In any embodiment, the applying step b) can include isolating miRNA from the sample to generate an isolated miRNA sample, and applying the isolated miRNA sample to the disk-coupled dots-on-pillar antenna (QMAX device) array.

In any embodiment, the QMAX device can contain a plurality of capture agents that each bind to a biomarker selected from Tables B1, B2, B3 and/or B7, wherein the reading step d) includes obtaining a measure of the amount of the plurality of biomarkers in the sample, and wherein the amount of the plurality of biomarkers in the sample is diagnostic of a disease or condition.

In any embodiment, the capture agent can be an antibody epitope and the biomarker can be an antibody that binds to the antibody epitope. In some embodiments, the antibody epitope includes a biomolecule, or a fragment thereof, selected from Tables B4, B5 or B6. In some embodiments, the antibody epitope includes an allergen, or a fragment thereof, selected from Table B5. In some embodiments, the antibody epitope includes an infectious agent-derived biomolecule, or a fragment thereof, selected from Table B6.

In any embodiment, the QMAX device can contain a plurality of antibody epitopes selected from Tables B4, B5 and/or B6, wherein the reading step d) includes obtaining a measure of the amount of a plurality of epitope-binding antibodies in the sample, and wherein the amount of the plurality of epitope-binding antibodies in the sample is diagnostic of a disease or condition.

In any embodiment, the sample can be an environmental sample, and wherein the analyte can be an environmental marker. In some embodiments, the environmental marker is selected from Table B8 in U.S. Provisional Application No. 62/234,538 and/or PCT Application No. PCT/US2016/054025.

In any embodiment, the method can include receiving or providing a report that indicates the safety or harmfulness for a subject to be exposed to the environment from which the sample was obtained.

In any embodiment, the method can include sending data containing the measured amount of the environmental marker to a remote location and receiving a report that indicates the safety or harmfulness for a subject to be exposed to the environment from which the sample was obtained.

In any embodiment, the QMAX device array can include a plurality of capture agents that each binds to an environmental marker selected from Table B8, and wherein the reading step d) can include obtaining a measure of the amount of the plurality of environmental markers in the sample.

In any embodiment, the sample can be a foodstuff sample, wherein the analyte can be a foodstuff marker, and wherein the amount of the foodstuff marker in the sample can correlate with safety of the foodstuff for consumption. In some embodiments, the foodstuff marker is selected from Table B9.

In any embodiment, the method can include receiving or providing a report that indicates the safety or harmfulness for a subject to consume the foodstuff from which the sample is obtained.

In any embodiment, the method can include sending data containing the measured amount of the foodstuff marker to a remote location and receiving a report that indicates the safety or harmfulness for a subject to consume the foodstuff from which the sample is obtained.

In any embodiment, the devices, apparatus, systems, and methods herein disclosed can include a plurality of capture agents that each binds to a foodstuff marker selected from Table B9 from in U.S. Provisional Application No. 62/234,538 and PCT Application No. PCT/US2016/054025, wherein the obtaining can include obtaining a measure of the amount of the plurality of foodstuff markers in the sample, and wherein the amount of the plurality of foodstuff marker in the sample can correlate with safety of the foodstuff for consumption.

Also provided herein are kits that find use in practicing the devices, systems and methods in the present invention.

The amount of sample can be about a drop of a sample. The amount of sample can be the amount collected from a pricked finger or fingerstick. The amount of sample can be the amount collected from a microneedle or a venous draw.

A sample can be used without further processing after obtaining it from the source, or can be processed, e.g., to enrich for an analyte of interest, remove large particulate matter, dissolve or resuspend a solid sample, etc.

Any suitable method of applying a sample to the QMAX device can be employed. Suitable methods can include using a pipette, dropper, syringe, etc. In certain embodiments, when the QMAX device is located on a support in a dipstick format, as described below, the sample can be applied to the QMAX device by dipping a sample-receiving area of the dipstick into the sample.

A sample can be collected at one time, or at a plurality of times. Samples collected over time can be aggregated and/or processed (by applying to a QMAX device and obtaining a measurement of the amount of analyte in the sample, as described herein) individually. In some instances, measurements obtained over time can be aggregated and can be useful for longitudinal analysis over time to facilitate screening, diagnosis, treatment, and/or disease prevention.

Washing the QMAX device to remove unbound sample components can be done in any convenient manner, as described above. In certain embodiments, the surface of the QMAX device is washed using binding buffer to remove unbound sample components.

Detectable labeling of the analyte can be done by any convenient method. The analyte can be labeled directly or indirectly. In direct labeling, the analyte in the sample is labeled before the sample is applied to the QMAX device. In indirect labeling, an unlabeled analyte in a sample is labeled after the sample is applied to the QMAX device to capture the unlabeled analyte, as described below.

### 4. Labels

The devices, apparatus, systems, and methods herein disclosed can be used with various types of labels, which include the labels disclosed, described and/or summarized in PCT Application (designating U.S.) No. PCT/US2016/045437, which was filed on Aug. 10, 2016, and is hereby incorporated by reference by its entirety.

In some embodiments, the label is optically detectable, such as but not limited to a fluorescence label. In some embodiments, the labels include, but are not limited to, IRDye8000 W, Alexa 790, Dylight 800, fluorescein, fluorescein isothiocyanate, succinimidyl esters of carboxyfluorescein, succinimidyl esters of fluorescein, 5-isomer of fluorescein dichlorotriazine, caged carboxyfluorescein-alanine-carboxamide, Oregon Green 488, Oregon Green 514; Lucifer Yellow, acridine Orange, rhodamine, tetramethylrhodamine, Texas Red, propidium iodide, JC-1 (5,5′,6,6′-tetrachloro-1,1′,3,3′-tetraethylbenzimidazoylcarbocyanine iodide), tetrabromorhodamine 123, rhodamine 6G, TMRM (tetramethyl rhodamine methyl ester), TMRE (tetramethyl rhodamine ethyl ester), tetramethylrosamine, rhodamine B and 4-dimethylaminotetramethylrosamine, green fluorescent protein, blue-shifted green fluorescent protein, cyan-shifted green fluorescent protein, red-shifted green fluorescent protein, yellow-shifted green fluorescent protein, 4-acetamido-4′-isothiocyanatostilbene-2,2′disulfonic acid; acridine and derivatives, such as acridine, acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-1-naphthyl)maleimide; anthranilamide; 4,4-difluoro-5-(2-thienyl)-4-bora-3a,4a diaza-5-indacene-3-propionic acid BODIPY; cascade blue; Brilliant Yellow; coumarin and derivatives: coumarin, 7-amino-4-methylcoumarin (AMC, Coumarin 120), 7-amino-4-trifluoromethylcoumarin (Coumarin 151); cyanine dyes; cyanosine; 4′,6-diaminidino-2-phenylindole (DAPI); 5′,5″-dibromopyrogallol-sulfonaphthalein (Bromopyrogallol Red); 7-diethylamino-3-(4′-isothiocyanatophenyl)-4-methylcoumarin; diethylenetriaamine pentaacetate; 4,4′-diisothiocyanatodihydro-stilbene-2,2′-disulfonic acid; 4,4′-diisothiocyanatostilbene-2,2′-disulfonic acid; 5-(dimethylamino]naphthalene-1-sulfonyl chloride (DNS, dansylchloride); 4-dimethylaminophenylazophenyl-4′-isothiocyanate (DABITC); eosin and derivatives: eosin, eosin isothiocyanate, erythrosin and derivatives: erythrosin B, erythrosin, isothiocyanate; ethidium; fluorescein and derivatives: 5-carboxyfluorescein (FAM), 5-(4,6-dichlorotriazin-2-yl)amino- -fluorescein (DTAF), 2′,7′dimethoxy-4′5′-dichloro-6-carboxyfluorescein (JOE), fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′-tetramethyl-6-carboxyrhodamine (TAMRA); tetramethyl rhodamine; tetramethyl hodamine isothiocyanate (TRITC); riboflavin; 5-(2′-aminoethyl)aminonaphthalene-1-sulfonic acid (EDANS), 4-(4′-dimethylaminophenylazo)benzoic acid (DABCYL), rosolic acid; CAL Fluor Orange 560; terbium chelate derivatives; Cy 3; Cy 5; Cy 5.5; Cy 7; IRD 700; IRD 800; La Jolla Blue; phthalo cyanine; and naphthalo cyanine, coumarins and related dyes, xanthene dyes such as rhodols, resorufins, bimanes, acridines, isoindoles, dansyl dyes, aminophthalic hydrazides such as luminol, and isoluminol derivatives, aminophthalimides, aminonaphthalimides, aminobenzofurans, aminoquinolines, dicyanohydroquinones, fluorescent europium and terbium complexes; combinations thereof, and the like. Suitable fluorescent proteins and chromogenic proteins include, but are not limited to, a green fluorescent protein (GFP), including, but not limited to, a GFP derived from Aequoria victoria or a derivative thereof, e.g., a “humanized” derivative such as Enhanced GFP; a GFP from another species such as Renilla reniformis, Renilla mulleri, or Ptilosarcus guernyi; “humanized” recombinant GFP (hrGFP); any of a variety of fluorescent and colored proteins from Anthozoan species; combinations thereof; and the like.

### 5. QMAX Device

The devices, apparatus, systems, and methods herein disclosed can include or use a QMAX device ((Q: quantification; M: magnifying; A: adding reagents; X: acceleration; also known as Q-card in some embodiments or compressed regulated open flow (CROF) device), which include the QMAX device listed, described and/or summarized in PCT Application (designating U.S.) Nos. PCT/US2016/045437 filed on Aug. 10, 2016, and U.S. Provisional Application No. 62,431,639 filed on Dec. 9, 2016 and 62/456,287 filed on Feb. 8, 2017, which are all hereby incorporated by reference by their entireties.

As used here, the terms “CROF Card (or card)”, “COF Card”, “QMAX-Card”, “Q-Card”, “CROF device”, “COF device”, “QMAX-device”, “CROF plates”, “COF plates”, and “QMAX-plates” are interchangeable, except that in some embodiments, the COF card does not comprise spacers; and the terms refer to a device that comprises a first plate and a second plate that are movable relative to each other into different configurations (including an open configuration and a closed configuration), and that comprises spacers (except some embodiments of the COF) that regulate the spacing between the plates. The term “X-plate” refers to one of the two plates in a CROF card, wherein the spacers are fixed to this plate. More descriptions of the COF Card, CROF Card, and X-plate are described in the provisional application Ser. No. 62/456,065, filed on Feb. 7, 2017, which is incorporated herein in its entirety for all purposes.

The term “compressed open flow (COF)” refers to a method that changes the shape of a flowable sample deposited on a plate by (i) placing other plate on top of at least a part of the sample and (ii) then compressing the sample between the two plates by pushing the two plates towards each other; wherein the compression reduces a thickness of at least a part of the sample and makes the sample flow into open spaces between the plates. The term “compressed regulated open flow” or “CROF” (or “self-calibrated compressed open flow” or “SCOF” or “SCCOF”) (also known as QMAX) refers to a particular type of COF, wherein the final thickness of a part or entire sample after the compression is “regulated” by spacers, wherein the spacers are placed between the two plates. Here the CROF device is used interchangeably with the QMAX card.

The term “open configuration” of the two plates in a QMAX process means a configuration in which the two plates are either partially or completely separated apart and the spacing between the plates is not regulated by the spacers

The term “closed configuration” of the two plates in a QMAX process means a configuration in which the plates are facing each other, the spacers and a relevant volume of the sample are between the plates, the relevant spacing between the plates, and thus the thickness of the relevant volume of the sample, is regulated by the plates and the spacers, wherein the relevant volume is at least a portion of an entire volume of the sample.

The term “a sample thickness is regulated by the plate and the spacers” in a QMAX process means that for a give condition of the plates, the sample, the spacer, and the plate compressing method, the thickness of at least a port of the sample at the closed configuration of the plates can be predetermined from the properties of the spacers and the plate.

The term “inner surface” or “sample surface” of a plate in a QMAX card refers to the surface of the plate that touches the sample, while the other surface (that does not touch the sample) of the plate is termed “outer surface”.

The term “height” or “thickness” of an object in a QMAX process refers to, unless specifically stated, the dimension of the object that is in the direction normal to a surface of the plate. For example, spacer height is the dimension of the spacer in the direction normal to a surface of the plate, and the spacer height and the spacer thickness means the same thing.

The term “area” of an object in a QMAX process refers to, unless specifically stated, the area of the object that is parallel to a surface of the plate. For example, spacer area is the area of the spacer that is parallel to a surface of the plate.

The term of QMAX card refers the device that perform a QMAX (e.g. CROF) process on a sample, and have or not have a hinge that connect the two plates.

The term “QMAX card with a hinge and “QMAX card” are interchangeable.

The term “angle self-maintain”, “angle self-maintaining”, or “rotation angle self-maintaining” refers to the property of the hinge, which substantially maintains an angle between the two plates, after an external force that moves the plates from an initial angle into the angle is removed from the plates.

In using QMAX card, the two plates need to be open first for sample deposition. However, in some embodiments, the QMAX card from a package has the two plates are in contact each other (e.g. a close position), and to separate them is challenges, since one or both plates are very thing. To facilitate an opening of the QMAX card, opening notch or notches are created at the edges or corners of the first plate or both places, and, at the close position of the plates, a part of the second plate placed over the opening notch, hence in the notch of the first plate, the second plate can be lifted open without a blocking of the first plate.

In the QMAX assay platform, a QMAX card uses two plates to manipulate the shape of a sample into a thin layer (e.g. by compressing). In certain embodiments, the plate manipulation needs to change the relative position (termed: plate configuration) of the two plates several times by human hands or other external forces. There is a need to design the QMAX card to make the hand operation easy and fast.

In QMAX assays, one of the plate configurations is an open configuration, wherein the two plates are completely or partially separated (the spacing between the plates is not controlled by spacers) and a sample can be deposited. Another configuration is a closed configuration, wherein at least part of the sample deposited in the open configuration is compressed by the two plates into a layer of highly uniform thickness, the uniform thickness of the layer is confined by the inner surfaces of the plates and is regulated by the plates and the spacers. In some embodiments, the average spacing between the two plates is more than 300 um.

In a QMAX assay operation, an operator needs to first make the two plates to be in an open configuration ready for sample deposition, then deposit a sample on one or both of the plates, and finally close the plates into a close position. In certain embodiments, the two plates of a QMAX card are initially on top of each other and need to be separated to get into an open configuration for sample deposition. When one of the plate is a thin plastic film (175 um thick PMA), such separation can be difficult to perform by hand. The present invention intends to provide the devices and methods that make the operation of certain assays, such as the QMAX card assay, easy and fast.

In some embodiments, the QMAX device comprises a hinge that connect two or more plates together, so that the plates can open and close in a similar fashion as a book. In some embodiments, the material of the hinge is such that the hinge can self-maintain the angle between the plates after adjustment. In some embodiments, the hinge is configured to maintain the QMAX card in the closed configuration, such that the entire QMAX card can be slide in and slide out a card slot without causing accidental separation of the two plates. In some embodiments, the QMAX device comprises one or more hinges that can control the rotation of more than two plates.

In some embodiments, the hinge is made from a metallic material that is selected from a group consisting of gold, silver, copper, aluminum, iron, tin, platinum, nickel, cobalt, alloys, or any combination of thereof. In some embodiments, the hinge comprises a single layer, which is made from a polymer material, such as but not limited to plastics. The polymer material is selected from the group consisting of acrylate polymers, vinyl polymers, olefin polymers, cellulosic polymers, noncellulosic polymers, polyester polymers, Nylon, cyclic olefin copolymer (COC), poly(methyl methacrylate) (PMMB), polycarbonate (PC), cyclic olefin polymer (COP), liquid crystalline polymer (LCP), polyamide (PB), polyethylene (PE), polyimide (PI), polypropylene (PP), poly(phenylene ether) (PPE), polystyrene (PS), polyoxymethylene (POM), polyether ether ketone (PEEK), polyether sulfone (PES), poly(ethylene phthalate) (PET), polytetrafluoroethylene (PTFE), polyvinyl chloride (PVC), polyvinylidene fluoride (PVDF), polybutylene terephthalate (PBT), fluorinated ethylene propylene (FEP), perfluoroalkoxyalkane (PFB), polydimethylsiloxane (PDMS), rubbers, or any combinations of thereof. In some embodiments, the polymer material is selected from polystyrene, PMMB, PC, COC, COP, other plastic, or any combination of thereof.

In some embodiments, the QMAX device comprises opening mechanisms such as but not limited to notches on plate edges or strips attached to the plates, making is easier for a user to manipulate the positioning of the plates, such as but not limited to separating the plates of by hand.

In some embodiments, the QMAX device comprises trenches on one or both of the plates. In certain embodiments, the trenches limit the flow of the sample on the plate.

### 6. Spacers

The devices, apparatus, systems, and methods herein disclosed can include or use a device (e.g. a QMAX device), which comprises spacers that are listed, described and/or summarized in PCT Application (designating U.S.) No. PCT/US2016/045437 filed on Aug. 10, 2016, and U.S. Provisional Application No. 62,431,639 filed on Dec. 9, 2016 and 62/456,287 filed on Feb. 8, 2017, which are all hereby incorporated by reference by their entireties.

In essence, the term “spacers” or “stoppers” refers to, unless stated otherwise, the mechanical objects that set, when being placed between two plates, a limit on the minimum spacing between the two plates that can be reached when compressing the two plates together. Namely, in the compressing, the spacers will stop the relative movement of the two plates to prevent the plate spacing becoming less than a preset (i.e. predetermined) value.

The term “a spacer has a predetermined height” and “spacers have a predetermined inter-spacer distance” means, respectively, that the value of the spacer height and the inter spacer distance is known prior to a QMAX process. It is not predetermined, if the value of the spacer height and the inter-spacer distance is not known prior to a QMAX process. For example, in the case that beads are sprayed on a plate as spacers, where beads are landed at random locations of the plate, the inter-spacer distance is not predetermined. Another example of not predetermined inter spacer distance is that the spacers moves during a QMAX processes.

The term “a spacer is fixed on its respective plate” in a QMAX process means that the spacer is attached to a location of a plate and the attachment to that location is maintained during a QMAX (i.e. the location of the spacer on respective plate does not change) process. An example of “a spacer is fixed with its respective plate” is that a spacer is monolithically made of one piece of material of the plate, and the location of the spacer relative to the plate surface does not change during the QMAX process. An example of “a spacer is not fixed with its respective plate” is that a spacer is glued to a plate by an adhesive, but during a use of the plate, during the QMAX process, the adhesive cannot hold the spacer at its original location on the plate surface and the spacer moves away from its original location on the plate surface.

### 7. Adaptor

The devices, apparatus, systems, and methods herein disclosed can be used with an adaptor, which is configured to accommodate the device and connect the device to a reader, such as but not limited to a smartphone. In some embodiments, the Q-cards are used together with sliders that allow the card to be inserted into the adaptor so that the card can be read by a smartphone detection system. The structure, material, function, variation, dimension and connection of the Q-card, the sliders, and the adaptor are disclosed, listed, described, and/or summarized in PCT Application (designating U.S.) Nos. PCT/US2016/045437 filed on Aug. 10, 2016 and PCT/US0216/051775 filed on Sep. 14, 2016, US Provisional Application Nos. 62/456,590 filed on Feb. 8, 2017, 62/459,554 filed on Feb. 15, 2017, and 62/460,075 filed on Feb. 8, 2017, all of which applications are incorporated herein in their entireties for all purposes.

In some embodiments, the adaptor comprises a receptacle slot, which is configured to accommodate the QMAX device when the device is in a closed configuration. In certain embodiments, the QMAX device has a sample deposited therein and the adaptor can be connected to a mobile device (e.g. a smartphone) so that the sample can be read by the mobile device. In certain embodiments, the mobile device can detect and/or analyze a signal from the sample. In certain embodiments, the mobile device can capture images of the sample when the sample is in the QMAX device and positioned in the field of view (FOV) of a camera, which in certain embodiments, is part of the mobile device.

In some embodiments, the adaptor comprises optical components, which are configured to enhance, magnify, and/or optimize the production of the signal from the sample. In some embodiments, the optical components include parts that are configured to enhance, magnify, and/or optimize illumination provided to the sample. In certain embodiments, the illumination is provided by a light source that is part of the mobile device. In some embodiments, the optical components include parts that are configured to enhance, magnify, and/or optimize a signal from the sample. The structures, functions, and configurations of the optical components in some embodiments can be found in PCT Application (designating U.S.) Nos. PCT/US2016/045437 filed on Aug. 10, 2016 and PCT/US0216/051775 filed on Sep. 14, 2016, US Provisional Application Nos. 62/456,590 filed on Feb. 8, 2017, 62/459,554 filed on Feb. 15, 2017, and 62/460,075 filed on Feb. 8, 2017, all of which applications are incorporated herein in their entireties for all purposes.

### 8. Dimensions

The devices, apparatus, systems, and methods herein disclosed can include or use a QMAX device, which can comprise plates and spacers. In some embodiments, the dimension of the individual components of the QMAX device and its adaptor are listed, described and/or summarized in PCT Application (designating U.S.) No. PCT/US2016/045437 filed on Aug. 10, 2016, and U.S. Provisional Application Nos. 62,431,639 filed on Dec. 9, 2016 and 62/456,287 filed on Feb. 8, 2017, which are all hereby incorporated by reference by their entireties.

In some embodiments, the dimensions are listed in the Tables below:

Plates:

| Para- | | Preferred |
|---|---|---|
| meters | Embodiments | Embodiments |
| Shape | round, ellipse, rectangle, | at least one of |
| | triangle, polygonal, ring- | the two (or |
| | shaped, or any superposition | more) plates of |
| | of these shapes; the | the QMAX |
| | two (or more) plates of the | card has round |
| | QMAX card can have | corners for |
| | the same size and/or shape, | user safety concerns, |
| | or different size and/or | wherein the |
| | shape; | round corners |
| | | have a diameter |
| | | of 100 um or |
| | | less, 200 um |
| | | or less, 500 um |
| | | or less, 1 mm |
| | | or less, 2 mm |
| | | or less, 5 mm |
| | | or less 10 mm |
| | | or less, 50 mm |
| | | or less, or in |
| | | a range between |
| | | any two of |
| | | the values. |
| Thickness | the average thickness for at | For at least one |
| | least one of the plates | of the plates |
| | is 2 nm or less, 10 nm | is in the range |
| | or less, 100 nm or less, | of 0.5 to 1.5 |
| | 200 nm or less, 500 nm | mm; around |
| | or less, 1000 nm or less, | 1 mm; in the |
| | 2 μm (micron) or less, | range of 0.15 |
| | 5 μm or less, 10 μm or | to 0.2 mm; or |
| | less, 20 μm or less, | around 0.175 mm |
| | 50 μm or less, 100 μm or | |
| | less, 150 μm or less, | |
| | 200 μm or less, 300 μm | |
| | or less, 500 μm or less, | |
| | 800 μm or less, 1 mm | |
| | (millimeter) or less, | |
| | 2 mm or less, 3 mm or less, | |
| | 5 mm or less, 10 mm | |
| | or less, 20 mm or less, | |
| | 50 mm or less, 100 mm | |
| | or less, 500 mm or less, | |
| | or in a range between any two of | |
| | these values | |
| Lateral | For at least one of the | For at least |
| Area | plate is 1 mm2 (square | one plate of the |
| | millimeter) or less, 10 | QMAX card |
| | mm2 or less, 25 mm2 or | is in the range |
| | less, 50 mm2 or less, | of 500 to |
| | 75 mm2 or less, 1 cm2 | 1000 mm²; or |
| | (square centimeter) or less, | around 750 mm². |
| | 2 cm2 or less, 3 cm2 or | |
| | less, 4 cm2 or less, 5 cm2 | |
| | or less, 10 cm2 or less, | |
| | 100 cm2 or less, 500 cm2 | |
| | or less, 1000 cm2 or less, | |
| | 5000 cm2 or less, 10,000 | |
| | cm2 or less, 10,000 cm2 | |
| | or less, or in a range | |
| | between any two of these | |
| | values | |
| Lateral | For at least one of the plates | For at least one |
| Linear | of the QMAX card is 1 | plate of the |
| Dimension | mm or less, 5 mm or less, | QMAX card |
| (width, | 10 mm or less, 15 mm or | is in the range |
| length, or | less, 20 mm or less, 25 mm | of 20 to 30 mm; |
| diameter, | or less, 30 mm or less, | or around |
| etc.) | 35 mm or less, 40 mm or less, | 24 mm |
| | 45 mm or less, 50 mm | |
| | or less, 100 mm or less, | |
| | 200 mm or less, 500 mm or | |
| | less, 1000 mm or less, | |
| | 5000 mm or less, or in a range | |
| | between any two of these values | |
| Recess | 1 um or less, 10 um or less, | In the range of |
| width | 20 um or less, 30 um or | 1 mm to 10 |
| | less, 40 um or less, 50 um | mm; Or |
| | or less, 100 um or less, | About 5 mm |
| | 200 um or less, 300 um or | |
| | less, 400 um or less, 500 | |
| | um or less, 7500 um or less, | |
| | 1 mm or less, 5 mm or | |
| | less, 10 mm or less, | |
| | 100 mm or less, or 1000 mm or | |
| | less, or in a range between | |
| | any two of these values. | |

Hinge:

| | | Preferred |
|---|---|---|
| Parameters | Embodiments | Embodiments |
| Number | 1, 2, 3, 4, 5, or more | 1 or 2 |
| Length of | 1 mm or less, 2 mm or less, | In the range |
| Hinge | 3 mm or less, 4 mm | of 5 mm |
| Joint | or less, 5 mm or less, 10 | to 30 mm. |
| | mm or less, 15 mm or | |
| | less, 20 mm or less, 25 | |
| | mm or less, 30 mm or less, | |
| | 40 mm or less, 50 mm or | |
| | less, 100 mm or less, | |
| | 200 mm or less, or 500 | |
| | mm or less, or in a range | |
| | between any two of these values | |
| Ratio | 1.5 or less, 1 or less, 0.9 or | In the range |
| (hinge | less, 0.8 or less, 0.7 or | of 0.2 |
| joint | less, 0.6 or less, 0.5 or less, | to 1; or |
| length | 0.4 or less, 0.3 or less, | about 1 |
| vs. | 0.2 or less, 0.1 or less, | |
| aligning | 0.05 or less or in a range | |
| plate edge | between any two of these | |
| length | values. | |
| Area | 1 mm² or less, 5 mm² or less, | In the |
| | 10 mm² or less, 20 | range of 20 |
| | mm² or less, 30 mm² or less, | to 200 mm²; |
| | 40 mm² or less, 50 | or about |
| | mm² or less, 100 mm² or less, | 120 mm² |
| | 200 mm² or less, 500 | |
| | mm² or less, or in a range | |
| | between any of the two | |
| | values | |
| Ratio (hinge | 1 or less, 0.9 or less, 0.8 or | In the range of |
| area vs. | less, 0.7 or less, 0.6 or | 0.05 to 0.2, |
| plate area) | less, 0.5 or less, 0.4 or less, | around |
| | 0.3 or less, 0.2 or less, | 0.15 |
| | 0.1 or less, 0.05 or less, 0.01 | |
| | or less or in a range | |
| | between any two of these values | |
| Max. Open | 15 or less, 30 or less, 45 or | In the range |
| Degree | less, 60 or less, 75 or | of 90 |
| | less, 90 or less, 105 or less, | to 180 degrees |
| | 120 or less, 135 or less, | |
| | 150 or less, 165 or less, | |
| | 180 or less, 195 or less, | |
| | 210 or less, 225 or less, | |
| | 240 or less, 255 or less, | |
| | 270 or less, 285 or less, | |
| | 300 or less, 315 or less, | |
| | 330 or less, 345 or less or | |
| | 360 or less degrees, or in | |
| | a range between any | |
| | two of these values | |
| No. of | 1, 2, 3, 4, 5, or more | 1 or 2 |
| Layers | | |
| Layer | 0.1 um or less, 1 um or less, | In the range |
| thickness | 2 um or less, 3 um or | of 20 |
| | less, 5 um or less, 10 um | um to |
| | or less, 20 um or less, 30 | 1 mm; or |
| | um or less, 50 um or less, | Around 50 um |
| | 100 um or less, 200 um | |
| | or less, 300 um or less, | |
| | 500 um or less, 1 mm or | |
| | less, 2 mm or less, and a | |
| | range between any two of | |
| | these values | |
| Angle- | Limiting the angle adjustment | No more |
| maintaining | with no more than | than ±2 |
| | ±90, ±45, ±30, ±25, ±20, | |
| | ±15, ±10, ±8, ±6, ±5, ±4, | |
| | ±3, ±2, or ±1, or in a | |
| | range between any two of | |
| | these values | |

Notch:

| | | Preferred |
|---|---|---|
| Parameters | Embodiments | Embodiments |
| Number | 1, 2, 3, 4, 5, or more | 1 or 2 |
| Shape | round, ellipse, rectangle, | Part of a circle |
| | triangle, polygon, ring- | |
| | shaped, or any superposition | |
| | or portion of these | |
| | shapes. | |
| Positioning | Any location along any | |
| | edge except the hinge | |
| | edge, or any corner joint | |
| | by non-hinge edges | |
| Lateral | 1 mm or less, 2.5 mm or | In the range of 5 |
| Linear | less, 5 mm or less, 10 | mm to 15 mm; |
| Dimension | mm or less, 15 mm or less, | or about 10 mm |
| (Length | 20 mm or less, 25 mm | |
| along the | or less, 30 mm or less, | |
| edge, | 40 mm or less, 50 mm or | |
| radius, etc.) | less, or in a range between | |
| | any two of these | |
| | values | |
| Area | 1 mm² (square millimeter) | In the range of 10 |
| | or less, 10 mm² or less, | to 150 mm²; or |
| | 25 mm² or less, 50 mm² | about 50 mm² |
| | or less, 75 mm² or less or | |
| | in a range between any | |
| | two of these values. | |

Trench:

| | | Preferred |
|---|---|---|
| Parameters | Embodiments | Embodiments |
| Number | 1, 2, 3, 4, 5, or more | 1 or 2 |
| Shape | Closed (round, ellipse, | |
| | rectangle, triangle, polygon, | |
| | ring-shaped, or any | |
| | superposition or portion of | |
| | these shapes) or open- | |
| | ended (straight line, curved | |
| | line, arc, branched tree, | |
| | or any other shape with | |
| | open endings); | |
| Length | 0.001 mm or less, 0.005 | |
| | mm or less, 0.01 mm or | |
| | less, 0.05 mm or less, 0.1 | |
| | mm or less, 0.5 mm or | |
| | less, 1 mm or less, 2 mm | |
| | or less, 5 mm or less, 10 | |
| | mm or less, 20 mm or | |
| | less, 50 mm or less, 100 mm | |
| | or less, or in a range | |
| | between any two of these | |
| | values | |
| Cross- | 0.001 mm² or less, 0.005 | |
| sectional | mm² or less, 0.01 mm² or | |
| Area | less, 0.05 mm² or less, 0.1 | |
| | mm² or less, 0.5 mm² or | |
| | less, 1 mm² or less, 2 mm² | |
| | or less, 5 mm² or less, 10 | |
| | mm² or less, 20 mm² or | |
| | less, or in a range between | |
| | any two of these values. | |
| Volume | 0.1 uL or more, 0.5 uL or | In the range of 1 |
| | more, 1 uL or more, 2 uL | uL to 20 uL; or |
| | or more, 5 uL or more, | About 5 uL |
| | 10 uL or more, 30 uL or | |
| | more, 50 uL or more, | |
| | 100 uL or more, 500 uL or | |
| | more, 1 mL or more, or | |
| | in a range between any two | |
| | of these values | |

Receptacle Slot

| | | Preferred |
|---|---|---|
| Parameters | Embodiments | Embodiments |
| Shape of | round, ellipse, rectangle, | |
| receiving | triangle, polygon, ring- | |
| area | shaped, or any superposition | |
| | of these shapes; | |
| Difference | 100 nm, 500 nm, 1 um, 2 um, | In the range of |
| between | 5 um, 10 um, 50 um, | 50 to 300 um; |
| sliding track | 100 um, 300 um, 500 um, | or about 75 um |
| gap size and | 1 mm, 2 mm, 5 mm, 1 | |
| card | cm, or in a range between | |
| thickness | any two of the values. | |
| Difference | 1 mm² (square millimeter) | |
| between | or less, 10 mm² or less, | |
| receiving | 25 mm² or less, 50 mm² or | |
| area and | less, 75 mm² or less, 1 | |
| card area | cm² (square centimeter) | |
| | or less, 2 cm² or less, 3 cm² | |
| | or less, 4 cm² or less, 5 cm² | |
| | or less, 10 cm² or less, | |
| | 100 cm² or less, or in a | |
| | range between any of the | |
| | two values. | |

### 9. Hand Pressing

For the devices, apparatus, systems, and methods herein disclosed, human hands can be used for manipulating or handling or the plates and/or samples. In some embodiments, human hands can be used to press the plates into a closed configuration; In some embodiments, human hands can be used to press the sample into a thin layer. The manners in which hand pressing is employed are described and/or summarized in PCT Application (designating U.S.) Nos. PCT/US2016/045437 filed on Aug. 10, 2016 and PCT/US0216/051775 filed on Sep. 14, 2016, and in US Provisional Application Nos. 62/431,639 filed on Dec. 9, 2016, 62/456,287 filed on Feb. 8, 2017, 62/456,065 filed on Feb. 7, 2017, 62/456,504 filed on Feb. 8, 2017, and 62/460,062 filed on Feb. 16, 2017, which are all hereby incorporated by reference by their entireties.

In some embodiments, human hand can be used to manipulate or handle the plates of the QMAX device. In certain embodiments, the human hand can be used to apply an imprecise force to compress the plates from an open configuration to a closed configuration. In certain embodiments, the human hand can be used to apply an imprecise force to achieve high level of uniformity in the thickness of the sample (e.g. less than 5%, 10%, 15%, or 20% variability).

### 10. Smartphone

The devices, apparatus, systems, and methods herein disclosed can be used with a mobile device, such as but not limited to a smartphone. The smartphone detection technology is herein disclosed, or listed, described, and/or summarized in PCT Application (designating U.S.) Nos. PCT/US2016/045437 and PCT/US0216/051775, which were respectively filed on Aug. 10, 2016 and Sep. 14, 2016, U.S. Provisional Application No. 62/456,065, which was filed on Feb. 7, 2017, U.S. Provisional Application No. 62/456,287, which was filed on Feb. 8, 2017, and U.S. Provisional Application No. 62/456,504, which was filed on Feb. 8, 2017, all of which applications are incorporated herein in their entireties for all purposes.

In some embodiments, the smartphone comprises a camera, which can be used to capture images or the sample when the sample is positioned in the field of view of the camera (e.g. by an adaptor). In certain embodiments, the camera includes one set of lenses (e.g. as in iPhone™ 6). In certain embodiments, the camera includes at least two sets of lenses (e.g. as in iPhone™ 7). In some embodiments, the smartphone comprises a camera, but the camera is not used for image capturing.

In some embodiments, the smartphone comprises a light source such as but not limited to LED (light emitting diode). In certain embodiments, the light source is used to provide illumination to the sample when the sample is positioned in the field of view of the camera (e.g. by an adaptor). In some embodiments, the light from the light source is enhanced, magnified, altered, and/or optimized by optical components of the adaptor.

In some embodiments, the smartphone comprises a processor that is configured to process the information from the sample. The smartphone includes software instructions that, when executed by the processor, can enhance, magnify, and/or optimize the signals (e.g. images) from the sample. The processor can include one or more hardware components, such as a central processing unit (CPU), an application-specific integrated circuit (ASIC), an application-specific instruction-set processor (ASIP), a graphics processing unit (GPU), a physics processing unit (PPU), a digital signal processor (DSP), a field-programmable gate array (FPGA), a programmable logic device (PLD), a controller, a microcontroller unit, a reduced instruction-set computer (RISC), a microprocessor, or the like, or any combination thereof.

In some embodiments, the smartphone comprises a communication unit, which is configured and/or used to transmit data and/or images related to the sample to another device. Merely by way of example, the communication unit can use a cable network, a wireline network, an optical fiber network, a telecommunications network, an intranet, the Internet, a local area network (LAN), a wide area network (WAN), a wireless local area network (WLAN), a metropolitan area network (MAN), a wide area network (WAN), a public telephone switched network (PSTN), a Bluetooth network, a ZigBee network, a near field communication (NFC) network, or the like, or any combination thereof.

In some embodiments, the smartphone is an iPhone™, an Android™ phone, or a Windows™ phone.

### 11. Cloud

The devices, apparatus, systems, and methods herein disclosed can be used with cloud storage and computing technologies. The related cloud technologies are herein disclosed, or listed, described, and summarized in PCT Application (designating U.S.) Nos. PCT/US2016/045437 and PCT/US0216/051775, which were respectively filed on Aug. 10, 2016 and Sep. 14, 2016, U.S. Provisional Application No. 62/456,065, which was filed on Feb. 7, 2017, U.S. Provisional Application No. 62/456,287, which was filed on Feb. 8, 2017, and U.S. Provisional Application No. 62/456,504, which was filed on Feb. 8, 2017, all of which applications are incorporated herein in their entireties for all purposes.

In some embodiments, the cloud storage and computing technologies can involve a cloud database. Merely by way of example, the cloud platform can include a private cloud, a public cloud, a hybrid cloud, a community cloud, a distributed cloud, an inter-cloud, a multi-cloud, or the like, or any combination thereof. In some embodiments, the mobile device (e.g. smartphone) can be connected to the cloud through any type of network, including a local area network (LAN) or a wide area network (WAN).

In some embodiments, the data (e.g. images of the sample) related to the sample is sent to the cloud without processing by the mobile device and further analysis can be conducted remotely. In some embodiments, the data related to the sample is processed by the mobile device and the results are sent to the cloud. In some embodiments, both the raw data and the results are transmitted to the cloud.

### Aspects:

1. A device for analyzing an analyte in a sample through selective lysing, comprising:
   a first plate, a second plate, and spacers, wherein
      v. the plates are movable relative to each other into different configurations, including an open configuration and a closed configuration;
      vi. each of the plates has, on its respective sample surface, a sample contact area for contacting the sample, wherein the sample comprises an analyte and a non-analyte cell; and
      vii. one or both of the plates comprise the spacers, and the spacers are fixed to the respective plates; and
      viii. the height of the spacers is configured, so that in a closed configuration of the plates, the analyte is not substantially lysed while the non-analyte cell is substantially lysed;
      wherein in the open configuration, the two plates are partially or entirely separated apart, the spacing between the plates is not regulated by the spacers, and the sample is deposited on one or both of the plates;
      wherein in the closed configuration, which is configured after deposition of the sample in the open configuration: the relevant volume of the sample is compressed by the two plates into a layer of highly uniform thickness, and the uniform thickness of the layer is confined by the sample contact surfaces of the plates and is regulated by the plates and the spacers.
2. A device for analyzing platelets in a blood sample through selective lysing, comprising:
   a first plate, a second plate, and spacers, wherein
      i. the plates are movable relative to each other into different configurations, including an open configuration and a closed configuration;
      ii. each of the plates has, on its respective a sample surface, a blood sample contact area for contacting the sample, wherein the sample comprises platelets and red blood cells (RBC); and
      iii. one or both of the plates comprise the spacers, and the spacers are fixed to the respective plates; and
      iv. the height of the spacers is configured, so that in a closed configuration of the plates, the platelet is not substantially lysed while the RBC is substantially lysed;
      wherein in the open configuration, the two plates are partially or entirely separated apart, the spacing between the plates is not regulated by the spacers, and the sample is deposited on one or both of the plates;
      wherein in the closed configuration, which is configured after deposition of the sample in the open configuration: the relevant volume of the sample is compressed by the two plates into a layer of highly uniform thickness, and the uniform thickness of the layer is confined by the sample contact surfaces of the plates and is regulated by the plates and the spacers.
3. The device of any prior Aspect, wherein the final sample thickness regulated by the spacers is larger than that of the analyte size while avoiding to substantially lyse the analyte.
4. The device of any prior Aspect, wherein the final sample thickness regulated by the spacers is about the same as that of the analyte size while avoiding to substantially lyse the analyte.
5. The device of any prior Aspect, wherein the final sample thickness regulated by the spacers is smaller than that of the analyte size while avoiding to substantially lyse the analyte.
6. The device of any prior Aspect, further comprising a lysing reagent on the respective sample contact area, wherein the lysing reagent configured to assist the substantial lysing at the closed configuration of the plates.
7. A method for analyzing an analyte in a sample through selective lysing, comprising:
   i. having a device of any prior Aspect;
   ii. depositing a sample in an open configuration, and
   iii. closing the plates into a closed-configuration.
8. A method for analyzing platelet in a blood sample through selective lysing, comprising:
   i. having a device of any prior Aspect;
   ii. depositing a blood sample in an open configuration, and
   iii. closing the plates into a closed-configuration.
9. A device for analyzing platelets in a blood sample through selective lysing, comprising:
   a first plate, a second plate, and spacers, wherein
      i. the plates are movable relative to each other into different configurations, including an open configuration and a closed configuration;
      ii. each of the plates has, on its respective a sample surface, a blood sample contact area for contacting the sample, wherein the sample comprises platelets and red blood cells (RBC); and
      iii. one or both of the plates comprise the spacers, and the spacers are fixed to the respective plates; and
      iv. the height of the spacers is configured, so that in a closed configuration of the plates, the platelet is not substantially lysed while the RBC is substantially lysed;
      wherein in the open configuration, the two plates are partially or entirely separated apart, the spacing between the plates is not regulated by the spacers, and the sample is deposited on one or both of the plates;
      wherein in the closed configuration, which is configured after deposition of the sample in the open configuration: the relevant volume of the sample is compressed by the two plates into a layer of highly uniform thickness, and the uniform thickness of the layer is confined by the sample contact surfaces of the plates and is regulated by the plates and the spacers.
10. The device, system, and method of any prior Aspect, wherein the non-analyte cell comprises a cell not including an analyzable analyte.
11. The device, system, and method of any prior Aspect, wherein the blood sample is a whole blood sample.
12. A device for analyzing platelets in a blood sample, comprising:
   a first plate, a second plate, and spacers, wherein
      i. the plates are movable relative to each other into different configurations, including an open configuration and a closed configuration;
      ii. each of the plates has, on its respective sample surface, a sample contact area for contacting a blood sample, wherein the blood sample comprises red blood cells (RBCs) and platelets; and
      iii. one or both of the plates comprise the spacers, and the spacers are fixed to the respective plates; and
      iv. one or both of the plates comprise, on the respective sample contact area, a layer of lysing agent, wherein the lysing agent is configured such that, in the closed configuration, a substantial fraction of the RBCs in a relevant volume of the sample are lysed by the lysing agent dissolved in the relevant volume, and a substantial fraction of the platelets in the relevant volume of the sample are not lysed,
      wherein in the open configuration, the two plates are partially or entirely separated apart, the spacing between the plates is not regulated by the spacers, and the sample is deposited on one or both of the plates;
      wherein in the closed configuration, which is configured after deposition of the sample in the open configuration: the relevant volume of the sample is compressed by the two plates into a layer of highly uniform thickness, and the uniform thickness of the layer is confined by the sample contact surfaces of the plates and is regulated by the plates and the spacers; and
      wherein the relevant volume of the sample is a partial or entire volume of the sample.
13. A system for analyzing platelets in a blood sample, comprising:
   (a) a device of any prior Aspect;
   (b) an imager, comprising a camera and a light source for imaging the platelets in the relevant volume of the sample; and
   (c) a processor, comprising electronics, signal processors, hardware and software for receiving and processing the images and identifying and analyzing the platelets in the images.
14. A system for analyzing platelets in a blood sample, comprising:
   (a) a device of any prior Aspect;
   (b) a mobile communication device comprising:
      i. one or a plurality of cameras for imaging the platelets in the sample;
      ii. electronics, signal processors, hardware and software for receiving and/or processing the image of the platelets and for remote communication; and
   (c) a light source from either the mobile communication device or an external source, wherein the light source is configured to provide illumination to the sample for imaging with the cameras.
15. A method of analyzing platelets in a blood sample, comprising:
   (a) obtaining a blood sample, which comprises red blood cells (RBCs) and platelets;
   (b) obtaining a first and second plates that are movable relative to each other into different configurations, including an open configuration and a closed configuration, wherein:
      iii. each plate, on its respective surface, has a sample contact area for contacting the sample, and
      iv. one or both of the plates comprise spacers that are fixed with a respective sample contact surface,
         wherein the spacers have a predetermined substantially uniform height, and at least one of the spacers is inside the sample contact area;
   (c) depositing the sample on one or both of the plates when the plates are in an open configuration,
      wherein in the open configuration the two plates are partially or entirely separated apart and the spacing between the plates is not regulated by the spacers;
   (d) after (c), bringing the two plates together and pressing the plates into a closed configuration;
   (e) while the plates are at the closed configuration, acquiring images of the platelets in a relevant volume of the sample; and
   (f) identifying and analyzing the platelets in the acquired images,
      wherein in the closed configuration: the relevant volume of the sample is compressed by the two plates into a layer of highly uniform thickness, the uniform thickness of the layer is confined by the sample surfaces of the two plates and is regulated by the spacers and the plates,
      wherein the height of the spacers is selected such that in the closed configuration, a substantial fraction of the RBCs of the sample in the relevant volume of the sample are lysed, and a substantial fraction of the platelets in the relevant volume of the sample are not lysed; and
      wherein the relevant volume of the sample is a partial or entire volume of the sample.
16. A method of analyzing platelets in a blood sample, comprising the:
   (a) obtaining a blood sample, which comprises red blood cells (RBCs) and platelets;
   (b) obtaining a first and second plates that are movable relative to each other into different configurations, including an open configuration and a closed configuration, wherein:
      iv. each plate, on its respective surface, has a sample contact area for contacting the sample,
      v. one or both of the plates comprise spacers that are fixed with a respective sample contact area, and
      vi. one or both of the plates comprise, on the respective sample contact area, a layer of lysing agent, wherein the lysing agent is configured such that, in the closed configuration, a substantial fraction of the RBCs in a relevant volume of the sample are lysed by the lysing agent that is dissolved in the relevant volume, and a substantial fraction of the platelets in the relevant volume of the sample are not lysed,
         wherein the spacers have a predetermined substantially uniform height, and at least one of the spacers is inside the sample contact area;
   (c) depositing the sample on one or both of the plates when the plates are in an open configuration,
      wherein in the open configuration the two plates are partially or entirely separated apart and the spacing between the plates is not regulated by the spacers;
   (d) after (c), bringing the two plates together and pressing the plates into a closed configuration;
   (e) while the plates are at the closed configuration, acquiring images of the platelets in the relevant volume of the sample; and
   (f) identifying and analyzing the platelets in the acquired images,
      wherein in the closed configuration: the relevant volume of the sample is compressed by the two plates into a layer of highly uniform thickness, the uniform thickness of the layer is confined by the sample surfaces of the two plates and is regulated by the spacers and the plates, and
      wherein the relevant volume of the sample is a partial or entire volume of the sample.
17. The device, system, or method of any prior Aspect, wherein at least one of the plates is transparent.
18. The device, system, or method of any prior Aspect, wherein one or both of the plates comprises, on the respective sample contact area, a dye that, upon contacting the sample, is dissolved in the sample and stains the platelets.
19. The device, system, or method of Aspect 18, wherein the dye is fluorescently labeled.
20. The device, system, or method of Aspect 18, wherein the dye is acridine orange (AO).
21. The device, system, or method of any prior Aspect, wherein the blood sample is stained before being analyzed.
22. The device, system, or method of any prior Aspect, wherein on one or both the sample contact areas, the respective plate further comprises a layer of a reagent.
23. The device, system, or method of Aspect 22, wherein the reagent facilitates: (a) the lysing of the RBCs and/or WBCs, and/or (b) the unlysing of platelets.
24. The device, system, or method of Aspect 22, wherein the reagent is used for bio/chemical assay of the platelets.
25. The device, system, or method of any prior Aspect, wherein the lysing agent is selected from the group consisting of: ammonium chloride, organic quaternary ammonium surfactants, cyanide salts, and any combination thereof.
26. The device, system, or method of any prior Aspect, wherein the substantial fraction is at least 51%, 60%, 70%, 80%, 90%, 95% or 99% of a component in the relevant volume of the sample.
27. The device, system, or method of any prior Aspect, wherein the thickness variation of the layer of highly uniform thickness over the lateral area of the relevant volume is equal to or less than 40%, 30%, 20%, 15%, 10%, 7%, 5%, 3%, or 1%, or in a range between any of the two values, wherein the thickness variation is relative to the average thickness of the lateral area.
28. The device, system, or method of any prior Aspect, wherein the area of the highly uniform layer is equal to or larger than 0.1 mm², 0.5 mm², 1 mm², 3 mm², 5 mm², 10 mm², 20 mm², 50 mm², 70 mm², 100 mm², 200 mm², 500 mm², 800 mm², 1000 mm², 2000 mm², 5000 mm², 10000 mm², 20000 mm², 50000 mm², or 100000 mm²; or in a range between any of the two values.
29. The device, system, or method of any prior Aspect, wherein the blood sample is diluted or undiluted whole blood.
30. The device, system, or method of any prior Aspect, wherein the blood sample is partial blood sample.
31. The device, system, or method of any prior Aspect, wherein the spacer height is equal to or less than 2 um, 1.9 um, 1.8 um, 1.7 um, 1.6 um, 1.5 um, 1.4 um, 1.3 um, 1.2 um, 1.1 um, 1.0 um, 0.9 um, 0.8 um, 0.7 um, 0.6 um, 0.5 um, 0.4 um, 0.3 um, or 0.2 um, or in a range between any of the two values.
32. The device, system, or method of any prior Aspect, wherein in the closed configuration, a substantial fraction of white blood cells (WBCs) in the relevant volume of the sample are lysed, and the spacer height is equal to or less than 1.0 um, 0.9 um, 0.8 um, 0.7 um, 0.6 um, 0.5 um, 0.4 um, 0.3 um, or 0.2 um, or in a range between any of the two values.
33. The system of any prior Aspect, further comprising:
(d) a housing configured to hold the sample and to be mounted to the mobile communication device.
34. The system of any prior Aspect, wherein the mobile communication device, the light source, and the housing are configured to provide bright-field illumination of the sample, acquire and/or process optical images of the platelets in the relevant volume of the sample.
35. The system of any prior Aspect, wherein the mobile communication device, the light source, and the housing are configured to provide fluorescent illumination of the sample, acquire and/or process fluorescent images of platelets that are fluorescently labeled in the relevant volume of the sample.
36. The system of any prior Aspect, wherein the housing comprises optics for facilitating the imaging and/or signal processing of the sample by the mobile communication device, and a mount configured to hold the optics on the mobile communication device.
37. The system of any of any prior Aspect, wherein the mobile communication device is configured to communicate test results to a medical professional, a medical facility or an insurance company.
38. The system of any prior Aspect, wherein the mobile communication device is further configured to communicate information on the subject with the medical professional, medical facility or insurance company.
39. The system of any prior Aspect, wherein the mobile communication device is configured to receive a prescription, diagnosis or a recommendation from a medical professional.
40. The system of any prior Aspect, wherein the mobile communication device communicates with the remote location via a wifi or cellular network.
41. The system of any prior Aspect, wherein the mobile communication device is a mobile phone.
42. The method of any prior Aspect, wherein the step (e) of acquiring the images is performed by a mobile communication device that comprises:
   i. one or a plurality of cameras for imaging the platelets in the sample;
   ii. electronics, signal processors, hardware and software for receiving and/or processing the image of the platelets and for remote communication; and
a light source from either the mobile communication device or an external source.
43. The method of any prior Aspect, wherein the step (e) of acquiring the images comprises:
   i. acquiring optical images of the platelets in the relevant volume of the sample; and/or
   ii. acquiring fluorescent images of fluorescently-labeled platelets in the relevant volume of the sample in fluorescence mode, wherein the platelets are fluorescently labeled by a fluorescent dye that is pre-loaded into the sample or coated on the sample contact area of one or both of the plates.
44. The method of any prior Aspect, wherein the step (f) of identifying and analyzing is performed by a mobile communication device that is configured to receive and/or process the image of the platelets.
45. The method of any prior Aspect, wherein the analyzing comprises counting the number of the platelets in a first area of the images.
46. The method of Aspect 45, wherein the analyzing further comprises calculating the concentration of platelet in the sample by:
   (1) determining the volume of the sample covered by the first area through timing the first area by the uniform height of the spacers; and
   (2) dividing the count number of the platelets in the first area by the volume determined in step (1).
47. The device, system, or method of any prior Aspect, wherein the spacers have:
   v. a shape of pillar with substantially uniform cross-section and a flat top surface;
   vi. a ratio of the width to the height equal or larger than one;
   vii. a filling factor of equal to 1% or larger; and
   viii. a product of the filling factor and the Young's modulus of the spacer is 2 MPa or larger,
   wherein the filling factor is the ratio of the spacer contact area to the total plate area.
48. The device, system, or method of any prior Aspect, wherein an average value of the uniform thickness of the layer is substantially the same as the uniform height of the spacer with a variation of less than 10%.
49. The device, system, or method of any prior Aspect, wherein in the closed configuration at least 90% of the RBCs are lysed and at least 90% of the platelets are not lysed.
50. The device, system, or method of any prior Aspect, wherein in the closed configuration at least 99% of the RBCs are lysed and at least 99% of the platelets are not lysed.
51. The device, system, or method of any prior Aspect, wherein the variation of the layer of uniform thickness is less than 30 nm.
52. The device, system, or method of any prior Aspect, wherein the layer of uniform thickness sample has a thickness uniformity of up to +/−5%.
53. The device, system, or method of any prior Aspect, wherein the spacers are pillars with a cross-sectional shape selected from round, polygonal, circular, square, rectangular, oval, elliptical, or any combination of the same.
54. The device, system, or method of any prior Aspect, wherein the spacers have:
   vi. a shape of pillar with substantially uniform cross-section and a flat top surface;
   vii. a ratio of the width to the height equal or larger than one;
   viii. a predetermined constant inter-spacer distance that is in the range of 10 μm to 200 μm;
   ix. a filling factor of equal to 1% or larger; and
   x. a product of the filling factor and the Young's modulus of the spacer is 2 MPa or larger.
   wherein the filling factor is the ratio of the spacer contact area to a total plate area.
55. The device, system, or method of any prior Aspect, wherein pressing the plates into the closed configuration is conducted either in parallel or sequentially, the parallel pressing applies an external force on an intended area at the same time, and the sequential pressing applies an external force on a part of an intended area and gradually move to other area.
56. The device, system, or method of any prior Aspect, wherein the blood sample is analyzed by:
   i. illuminating at least part of the blood sample in the layer of uniform thickness;
   ii. obtaining one or more images of the cells using a CCD or CMOS sensor;
   iii. identifying the platelets in the image using a computer; and
   iv. counting a number of platelets in an area of the image.
57. The device, system, or method of any prior Aspect, wherein the layer of uniform thickness sample has a thickness uniformity of up to +/−5%.

We claim:

1. A device for analyzing an analyte in a sample through selective lysing, comprising:
a first plate, a second plate, and spacers, wherein
i. the plates are movable relative to each other into different configurations, including an open configuration and a closed configuration;
ii. each of the plates has, on its respective sample surface, a sample contact area for contacting the sample, wherein the sample comprises an analyte and a non-analyte cell; and
iii. one or both of the plates comprise the spacers, and the spacers are fixed to the respective plates; and
iv. the height of the spacers is configured, so that in a closed configuration of the plates, the analyte is not substantially lysed while the non-analyte cell is substantially lysed;
wherein in the open configuration, the two plates are partially or entirely separated apart, the spacing between the plates is not regulated by the spacers, and the sample is deposited on one or both of the plates;
wherein in the closed configuration, which is configured after deposition of the sample in the open configuration: the relevant volume of the sample is compressed by the two plates into a layer of highly uniform thickness, and the uniform thickness of the layer is confined by the sample contact surfaces of the plates and is regulated by the plates and the spacers.

2. A device for analyzing platelets in a blood sample through selective lysing, comprising:
a first plate, a second plate, and spacers, wherein
i. the plates are movable relative to each other into different configurations, including an open configuration and a closed configuration;
ii. each of the plates has, on its respective sample surface, a blood sample contact area for contacting the sample, wherein the sample comprises platelets and red blood cells (RBC); and
iii. one or both of the plates comprise the spacers, and the spacers are fixed to the respective plates; and
iv. the height of the spacers is configured, so that in a closed configuration of the plates, the platelet is not substantially lysed while the RBC is substantially lysed;
wherein in the open configuration, the two plates are partially or entirely separated apart, the spacing between the plates is not regulated by the spacers, and the sample is deposited on one or both of the plates;
wherein in the closed configuration, which is configured after deposition of the sample in the open configuration: the relevant volume of the sample is compressed by the two plates into a layer of highly uniform thickness, and the uniform thickness of the layer is confined by the sample contact surfaces of the plates and is regulated by the plates and the spacers.

3. The device of claim 1, wherein the final sample thickness regulated by the spacers is larger than that of the analyte size while avoiding to substantially lyse the analyte.

4. The device of claim 1, wherein the final sample thickness regulated by the spacers is about the same as that of the analyte size while avoiding to substantially lyse the analyte.

5. The device of claim 1, wherein the final sample thickness regulated by the spacers is smaller than that of the analyte size while avoiding to substantially lyse the analyte.

6. The device of claim 1, further comprising a lysing reagent on the respective sample contact area, wherein the lysing reagent configured to assist the substantial lysing at the closed configuration of the plates.

7. A method for analyzing an analyte in a sample through selective lysing, comprising:
i. having the device of claim 1;
ii. depositing a sample in an open configuration, and
iii. closing the plates into a closed-configuration.

8. A method for analyzing platelet in a blood sample through selective lysing, comprising:
i. having the device of claim 2;
ii. depositing a blood sample in an open configuration, and
iii. closing the plates into a closed-configuration.

9. The device of claim 1, wherein the non-analyte cell comprises a cell not including an analyzable analyte.

10. The device of claim 2, wherein the blood sample is a whole blood sample.

11. A device for analyzing platelets in a blood sample, comprising: a first plate, a second plate, and spacers, wherein
i. the plates are movable relative to each other into different configurations, including an open configuration and a closed configuration;
ii. each of the plates has, on its respective sample surface, a sample contact area for contacting a blood sample, wherein the blood sample comprises red blood cells (RBCs) and platelets; and
iii. one or both of the plates comprise the spacers, and the spacers are fixed to the respective plates; and
iv. one or both of the plates comprise, on the respective sample contact area, a layer of lysing agent, wherein the lysing agent is configured such that, in the closed configuration, a substantial fraction of the RBCs in a relevant volume of the sample are lysed by the lysing agent dissolved in the relevant volume, and a substantial fraction of the platelets in the relevant volume of the sample are not lysed,
wherein in the open configuration, the two plates are partially or entirely separated apart, the spacing between the plates is not regulated by the spacers, and the sample is deposited on one or both of the plates;
wherein in the closed configuration, which is configured after deposition of the sample in the open configuration: the relevant volume of the sample is compressed by the two plates into a layer of highly uniform thickness, and the uniform thickness of the layer is confined by the sample contact surfaces of the plates and is regulated by the plates and the spacers; and
wherein the relevant volume of the sample is a partial or entire volume of the sample.

12. A system for analyzing platelets in a blood sample, comprising:
(a) the device of claim 2;
(b) an imager, comprising a camera and a light source for imaging the platelets in the relevant volume of the sample; and
(c) a processor, comprising electronics, signal processors, hardware and software for receiving and processing the images and identifying and analyzing the platelets in the images.

13. A system for analyzing platelets in a blood sample, comprising:
(a) the device of claim 2;
(b) a mobile communication device comprising:
i. one or a plurality of cameras for imaging the platelets in the sample;
ii. electronics, signal processors, hardware and software for receiving and/or processing the image of the platelets and for remote communication; and
(c) a light source from either the mobile communication device or an external source, wherein the light source is configured to provide illumination to the sample for imaging with the cameras.

14. The device of claim 1, wherein at least one of the plates is transparent.

15. The device of claim 1, wherein one or both of the plates comprises, on the respective sample contact area, a dye that, upon contacting the sample, is dissolved in the sample and stains the platelets.

16. The device of claim 18, wherein the dye is fluorescently labeled.

17. The device of claim 18, wherein the dye is acridine orange (AO).

18. The device of claim 2, wherein the blood sample is stained before being analyzed.

19. The device of claim 2, wherein on one or both the sample contact areas, the respective plate further comprises a layer of a reagent.

20. The device of claim 22, wherein the reagent facilitates: (a) the lysing of the RBCs and/or WBCs, and/or (b) the unlysing of platelets.
